(19) United States
(12) Patent Application Publication
(10) Pub. No.: US 2010/0247578 A1
(43) Pub. Date: Sep. 30, 2010

(54) CARRIER SYSTEM FOR BIOLOGICAL AGENTS CONTAINING ORGANOSILICON COMPOUNDS AND USES THEREOF

(76) Inventor: Zoser B. Salama, Ravensburg (DE)

Correspondence Address: JOYCE VON NATZMER;PEQUIGNOT + MYERS LLC, 200 Madison Avenue, Suite 1901, New York, NY, 10016, US

(21) Appl. No.: 12/744,119

(22) PCT Filed: Aug. 17, 2009
(86) PCT No.: PCT/EP09/06071

§ 371 Date: May 21, 2010

(30) Foreign Application Priority Data
Aug. 15, 2008 (EP) 08075708.1

(51) Int. Cl. A61K 39/35; C07F 7/02; C07H 1/00; C07K 16/00; C07H 21/00; C12N 15/00; C07K 14/00; C07K 2/00; C07K 5/08; A61K 39/00

(52) U.S. Cl. 424/275.1; 556/400; 536/1.11; 530/391.1; 536/23.1; 435/320.1; 530/350; 530/300; 530/331; 424/184.1

(57) ABSTRACT

The invention relates to a novel organosilicon carrier system for biological agents that is produced via a simple, stable and reproducible preparation process that is capable of maintaining tertiary protein structure and biological activity of the proteins and/or other biological agents in the mixture, whereby the carrier system is obtainable by mixing one or more organosilicon compounds, selected from the group comprising organosilicon, sugar organosilicon, amino sugar organosilicon compounds, their derivatives, salts and/or the vesicles formed from them, with one or more biological agents, selected from the group comprising antigens, preantigens, antigen conjugates, antibodies, pre-antibodies, antibody conjugates, allergens, allergen extracts, nucleic acids, plasmids, proteins, peptides, pharmaceutical agents, immunologically active substances and/or cosmetics, in solution at a pH value between 7 and 8, preferably 7.4, followed by homogenisation or sonication of the mixture, followed by sterile filtration of the mixture, followed by lyophilisation.

The invention relates to a novel organosilicon carrier system for biological agents that is produced via a simple, stable and reproducible preparation process that is capable of maintaining tertiary protein structure and biological activity of the proteins and/or other biological agents in the mixture, whereby the carrier system is obtainable by mixing one or more organosilicon compounds, selected from the group comprising organosilicon, sugar organosilicon, amino sugar organosilicon compounds, their derivatives, salts and/or the vesicles formed from them, with one or more biological agents, selected from the group comprising antigens, pre-antigens, antigen conjugates, antibodies, pre-antibodies, antibody conjugates, allergens, allergen extracts, nucleic acids, plasmids, proteins, peptides, pharmaceutical agents, immunologically active substances and/or cosmetics, in solution at a pH value between 7 and 8, preferably 7.4, followed by homogenisation or sonication of the mixture, followed by sterile filtration of the mixture, followed by lyophilisation.

### BACKGROUND OF THE INVENTION

Many chemical entities have, due to various attributes, poor biological absorption (for example due to high molecular mass) or poor membrane permeability (due to high hydrophillicity). In such cases the use of a drug transport system is beneficial. The entrapment or encapsulation of bioactive or pharmaceutical agents within vesicles can assist in the delivery of these agents to cells and tissues in vivo. Liposomes and siosomes represent two such vesicles commonly used as drug delivery systems. Liposomes are typically phospholipid vesicles capable of encapsulating various biological agents, whereas siosomes are non-phospholipid vesicles created from organosilicon compounds with hydrophilic and hydrophobic ends.

Such delivery molecules can alter the bio-distribution and rate of delivery of an encapsulated bioactive agent in a number of ways. For example, drugs encapsulated in liposomes or siosomes are protected from interactions with serum factors which may chemically degrade the drug. The size of the liposome compared to the free drug also affects its access to certain sites in the body; this property can be advantageous in limiting drug delivery to certain sites.

A drug targeting system that hides molecules from the in vivo biological environment can also allow these molecules to cross into the intestinal endothelium or the blood brain barrier. Packaging a chemical entity in a molecule resembling a biological structure can solve some of these problems.

The use of organosilicon molecules in place of phospholipids provides multiple advantages. Organosilicon molecules are more stable at high temperatures, and the vesicles formed from them (siosomes) demonstrate significant advantages in regards to the stability of vesicular entrapments. Liposomes, or other vesicles such as virosomes or niosomes, have the disadvantages of low encapsulation efficiency and poor stability at high temperatures and/or levels of light exposure. Moreover they are not easily reproducible in a defined chemical composition. This results in an unacceptable variability of final product when preparing pharmaceutical agents using liposome particles.

The long chain di(acyloxy)dialkoxy-silanes and tetra(acyloxy)silanes, a method to prepare them, a method using them to prepare vesicles, and siosome vesicles consisting of long-chain di(acyloxy)dialkylsilanes and their use, have been described in the following patents: EP 0483465 B1 “Long chain di(alkoxy)dialkysilanes, di(alkoxy)diarysilanes, and tetra(alkoxy)silanes, methods to prepare them, vesicles prepared from them (Siosomes) and their use as vesicles for active substances”, DE 102005053011 A1 “Tetra organic silicone compounds, their use, their use for preparation of vesicles, vesicles prepared from them (Siosomes) and using them as active agents, as vesicles for active agents and for the preparation of vesicles”, and PCT/DE2006/001948 “Use of Tetraorganosilicon compounds”.

Despite success as drug delivery systems, the traditional liposome and siosome entrapments and encapsulations as disclosed in the prior art are severely limited in regards to production time and efficiency, the kinds of molecules that can be entrapped or encapsulated, and the stability of the final products. These vesicles are also usually limited to the encapsulation of only one active agent. This limitation has disadvantages in the use of vesicles as carriers for more than one agent and/or preparation of vaccines, because it is difficult to encapsulate a mixture of biologically active substances.

According to the relevant prior art, the preparation process for propanolol-encapsulated siosomes entails the following: an aqueous solution of propanolol, which has been brought to and kept at a temperature of 60 degrees, is gradually mixed with an ethanol silane solution over a period of 3 to 4 hours. The spontaneously forming vesicles then encapsulate the propanolol substance. In order to separate the non-encapsulated propanolol, dialysis using an isotonic potassium chloride solution is required until the point where no propanolol can be detected in solution. Encapsulation of insulin is carried out in a similar manner.

Although suitable for some pharmaceutical substances, a vast number of biologically active substances that require maintenance of three-dimensional structure, such as proteins or enzymes, cannot withstand such preparation conditions. The final functional structure of a single protein molecule, known as “tertiary structure”, is generally stabilized by non-local interactions, most commonly the formation of a hydrophobic core, but also through salt bridges, hydrogen bonds, disulfide bonds, and even post-translational modifications. The term “tertiary structure formation” is often used as synonymous with the term “protein folding”. Tertiary structure is what controls the basic form, and thus function, of any given protein. To be biologically active, proteins must adopt specific three dimensional tertiary structures.

Biologically active proteins unfold and lose their active state when exposed to denaturing agents. The denaturation of proteins involves the disruption and possible destruction of both secondary and tertiary structures. Denaturation disrupts the normal alpha-helix and beta sheets in a protein and uncoils it into a random shape. In tertiary structure there are four types of bonding interactions between “side chains” including hydrogen bonding, salt bridges, disulfide bonds, and non-polar hydrophobic interactions.

Heat disrupts hydrogen bonds and non-polar hydrophobic interactions. Increased temperature will affect interactions of tertiary structures. Increased flexibility, water binding and viscosity of solution and structures will be different from native protein as the result of the thermal denaturation, with the consequences of splitting of disulfide bonds and chemical alteration of amino residues. Upon cooling, aggregation and loss of solubility of the proteins may occur. Alcohol denatures proteins by disrupting the side chain intra-molecular hydrogen bonding. Hydrogen bonding occurs between amide groups in the secondary protein structure. Hydrogen bonding between “side chains” occurs in tertiary protein structure in a variety of amino acid combinations. All of these are disrupted by the addition of alcohol. Acids and bases disrupt salt bridges. Salt bridges result from the neutralization of an acid and amine on side chains. Additionally, reducing agents disrupt disulfide bonds, which are formed by the oxidation of the sulfhydryl groups on cysteine.

The presence or application of any of the above mentioned potentially denaturing conditions produces undesirable effects when preparing pharmaceutical compounds containing sensitive substances, such as proteins or enzymes.

There has been no organosilicon carrier system described in the prior art, capable of targeting or delivery of biological substances, which can be produced via a process amenable to maintaining tertiary protein structure.

This is particularly relevant in the preparation and administration of allergens and vaccines. Vaccines convey antigens from living or killed microorganisms (or proteins molecules derived from these antigens) to elicit immune responses. Antibodies and T-cells recognize particular parts of antigens, the epitopes, and not the whole organism or toxin. Importantly, it is the three dimensional structure of the epitope which is recognized by antibodies, thus it is beneficial that protein structure and folding be maintained during the preparation of vaccines. The application of vaccines also benefits from the controlled release and distribution provided by the use of organosilicon molecules.

This also applies for allergens. Allergens can be used to test a patient for hypersensitivity to specific substances. They can also be used to desensitize or hyposensitize allergic individuals. A large number of allergens are proteins, with an enormous range of molecular weight. As with vaccines, allergens have a localized region on their surface that is capable of eliciting an immune response. Most of these epitopes that are recognized by antibodies require three dimensional surface features, thus it is beneficial that protein structure and folding be maintained during the preparation of allergens for the desensitization of patients.

During the encapsulation of pharmaceutical agents, for example in both siosomes and liposomes, after loading the carrier systems the unbound materials are washed away by dialysis which results in a great loss of the often expensive biological agent. This also results in an unpredictable and high variability of the content of the encapsulated compounds. Additionally this can result in a risk for contamination of the environment. Furthermore, a safety risk regarding human use is introduced, because the final product does not have an accurate composition and is impossible to adjust.

### SUMMARY OF THE INVENTION

The technical problem underlying the present invention is to provide a novel carrier and targeting system for biological agents using organosilicon compounds that overcomes the disadvantages of the prior art.

This problem is solved by the features of the independent claims.

Preferred embodiments of the present invention are provided by the dependent claims.

The objective of the present invention is to provide a novel carrier and targeting system using organosilicon components for biological agents that are produced via a simple, stable and reproducible preparation process that is capable of maintaining tertiary protein structure, biologically relevant three-dimensional conformations and biological activity of the proteins and/or other biological agents in the mixture. Such carrier systems comprise of organosilicon compounds that bind a wide range of biological agents and ultimately mask the biological agent and mimic a biological membrane in vivo.

Therefore, an object of the invention is to provide carrier systems for biological agents obtainable by:
a) mixing one or more organosilicon compounds, selected from the group comprising:
   Organosilicon, sugar organosilicon, amino sugar organosilicon compounds, their derivatives, salts and/or the vesicles formed from them, with
   one or more biological agents, selected from the group comprising:
   Antigens, pre-antigens, antigen conjugates, antibodies, pre-antibodies, antibody conjugates, allergens, allergen extracts, nucleic acids, plasmids, proteins, peptides, pharmaceutical agents, immunologically active substances and/or cosmetics,
in solution at a pH value between 7 and 8, preferably 7.4, followed by
b) Homogenisation or sonication of the mixture, followed by
c) Sterile filtration of the mixture, followed by
d) Lyophilisation.

The functional interplay of the steps involved in the method of manufacture leads to a synergistic effect, for example the formation of complexes between the organosilicon molecules and biological agents in their native structure. It was unexpected that the resulting carrier system complexes could lead to the following surprising advantages, such as improved bio-distribution in a patient, reduced in vivo degradation of complexed biological agents, longer release times and thus fewer side effects for patients.

The combination of these manufacture steps produces a carrier system complex that is unexpectedly more stable than the prior art siosome encapsulations, that is easier to produce and that maintains tertiary protein structure. The manufacture steps required for the production of the claimed carrier system are functionally linked, and must occur in the provided sequence for the final product to demonstrate the advantageous features as described herein. Despite each of the manufacture steps being known in the art, the specific combination and order of the steps involved in the manufacture process has been neither described nor alluded to in the prior art, and leads to the production of a carrier system with surprising advantages and properties, as are described herein. Importantly, the method of manufacture represents a surprisingly simple solution to the problem of the invention, and replaces a more complex set of techniques disclosed in the prior art.

In a preferred embodiment the carrier system of the present invention is intended to incorporate organosilicon, sugar organosilicon, or aminosugar organosilicon compounds of the general formula 1:

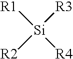

Where R1, R2, R3 and R4 can be the same or different

Possible R Group Identities
Alkyl or aryl residues
Fatty acids
Aromatic or aliphatic heterocycles
Sugar residues
Peptide residues (with free or protected amino groups)

R1 and R2, which can be the same or different, each represent an acyloxy residue of the formula R—COO— or a peptide residue of the formula

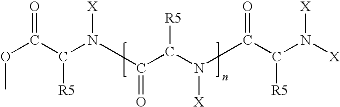

wherein R represents an unbranched or branched alkyl, alkenyl or alkinyl residue with 5 to 29 C atoms which can be substituted by one to three halogen atoms, alkoxy residues with 1 to 18 C atoms or amino groups, the residues R5, which can be the same or different, represent the residue remaining after the removal of the group

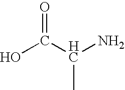

from an amino acid occurring in nature, the residues X, which may be the same or different, represent a hydrogen atom or an amino-protective group usually occurring in peptide chemistry and n represents an integer from 1 to 12 and R3 and R4 which can be the same or different, each represent an aryl group, such as a phenyl group, or the residue remaining after the removal of a hydrogen atom from a monosaccharide, disaccharide, amino sugar or a hydroxyl carbon acid, or alkoxy residues with 1-5 C atoms, or acyl residues of an amino acid occurring in nature with a free or protected amino group, or a dipeptide, tripeptide or tetrapeptide residue of an amino acid occurring in nature with free or protected amino groups, or they have the meaning R1 and R2, with R representing an unbranched or branched alkyl, akenyl or alkinyl residue.

Unexpectedly the organosilicon, sugar organosilicon, amino sugar organosilicon compounds and derivatives of the general formula 1 form complexes with the proteins, antigens, and the other biological agents and mixtures claimed in this patent through appropriate selection of the preparation conditions. The particular organosilicon compounds of the general formula 1, particularly the sugar organosilicons and amino sugar organosilicons, form surprisingly stable carrier system complexes.

The present invention utilises organosilicon compounds, according to the general formula 1, in providing a novel carrier system for biological agents not made exclusively of vesicles, but rather of a complex combination of organosilicon compounds with biological agents. The resulting carrier system complexes of the present invention differ significantly from the siosomes and liposomes described in the prior art. The prior art disclosure of organosilicon compounds is focused on encapsulation and entrapment of various biological agents. Of primary importance for siosomes or liposomes is the formation of vesicles in which the biological agents are entrapped or encapsulated. Such vesicles, with or without encapsulated or entrapped agents, are structurally distinct from the carrier system complexes of the present invention. Vesicles are typically not formed under the method of manufacture conditions of the present invention.

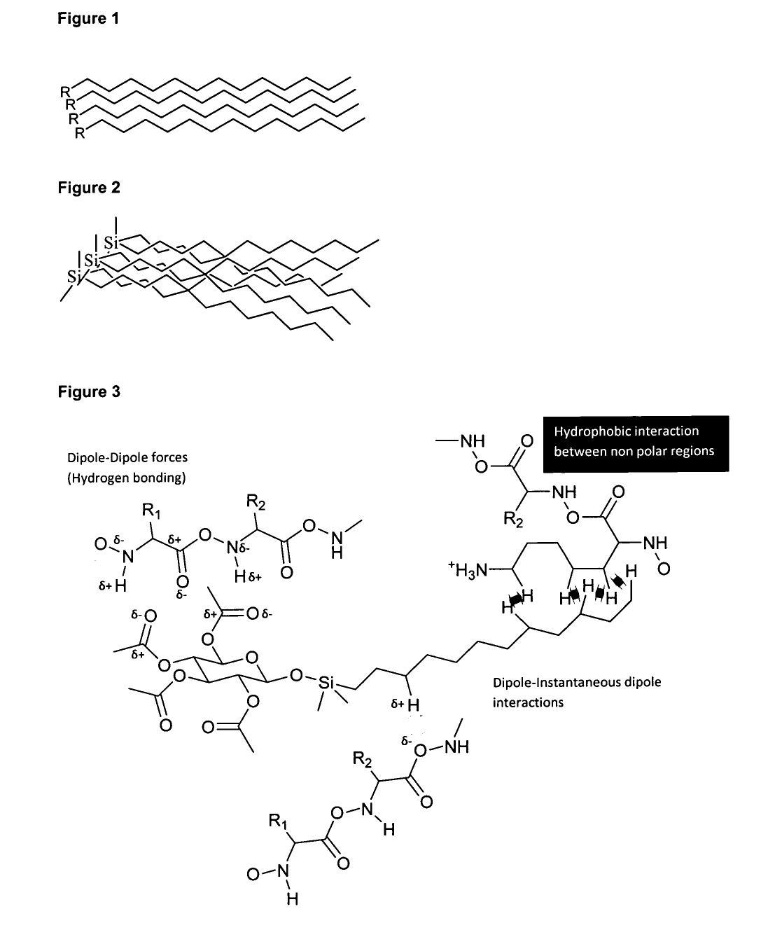

The binding of the organosilicon compounds to the biologically active substance within the carrier system of the present invention involves the formation of multiple hydrogen and non-covalent bonds between the biological substance itself and the organosilicon residues such as alkyl, aryl, peptides and fatty acids (See FIGS. 1-3). Considered individually the attractive forces (hydrogen and electrostatic bonds, Van der Waal's and hydrophobic forces) are weak in comparison to covalent bonds. However the large number of interactions results in a large total binding energy. In addition, partial encapsulation, entrapment and/or adsorption of the antigens, proteins, immunologically active substances and/or other biological agents in the sugar organosilicon compounds and/or its vesicles acts as a mimic to the natural physiological compounds of the cell membrane.

The following non-covalent intermolecular forces are involved in the formation of the complex combinations of the carrier systems of the present invention, between the organosilicon, sugar organosilicon, amino sugar organosilicon compounds of the present invention and the antigens, pre-antigens, antigen conjugates, antibodies, pre-antibodies, antibody conjugates, nucleic acids, plasmids, proteins, peptides, allergens, allergen extracts, pharmaceutical agents, immunologically active substances and/or cosmetics.
1. Hydrophobic interactions between non-polar regions involve for example the hydrophobic fatty acid chains of the organosilicon, sugar organosilicon, amino sugar organosilicon compounds and siosomes.
2. Non-specific protein-protein interactions between protein segments of an allergen and/or the peptide residues of the organosilicon, sugar organosilicon, amino sugar organosilicon compounds.
3. Protein-saccharide interactions; however, there is evidence to suggest that the major forces behind these interactions are electrostatic in nature.
4. Van der Waals interactions including:
   a. Dipole-dipole interactions, such as the hydrogen bonds between the O—H and the N—H bonds of the saccharides, amino residues, peptide residues of the organosilicon, sugar organosilicon, amino sugar organosilicon compounds, and the proteins, allergen protein mixtures, nucleic acids and/or antibodies.
   b. Dipole-induced dipole interactions; for example H₂O and saccharide residues and/or peptide residues in the organosilicon or sugar organosilicon molecules.
   c. Dispersion forces (London forces); including induced dipole-induced dipole forces, for example between two aliphatic hydrogens, for example in the organosilicon, sugar organosilicon, amino sugar organosilicon compounds and peptide residues of the proteins in the mixture.
5. Electrostatic interactions between charged residues, for example peptide residues and organosilicon, sugar organosilicon, amino sugar organosilicon compounds, and salts.
6. Ion-dipole forces; for example those between the salts of the buffer used in the preparation of the complex and H₂O.
7. Ion-ion forces; for example those involving the salts of the buffer solution.

Furthermore, it is a preferred embodiment of the present invention to provide carrier systems whereby one or more of the organosilicon, sugar organosilicon and/or amino sugar organosilicon compounds is optionally covalently attached to one or more of the antigens, antibodies and/or other biological agents. The covalent attachment of the organosilicon component to an antibody leads surprisingly to greater stability of carrier system formation, and surprisingly to no loss of the biological activity of the antibody. This embodiment is intended especially for the targeting of carrier system complexes to specific epitopes, as may be displayed by specific cell types in the body of the patient.

A further object of the invention is to provide a method for producing the carrier system of the present invention, characterised by
a) mixing one or more organosilicon compounds, selected from the group comprising:
   Organosilicon, sugar organosilicon, amino sugar organosilicon compounds, their derivatives, salts and/or the vesicles formed from them, with
   one or more biological agents, selected from the group comprising: antigens, pre-antigens, antigen conjugates, antibodies, pre-antibodies, antibody conjugates, allergens, allergen extracts, nucleic acids, plasmids, proteins, peptides, pharmaceutical agents, immunologically active substances and/or cosmetics,
in solution at a pH value between 7 and 8, preferably 7.4, followed by
b) Homogenisation or sonication of the mixture, followed by
c) Sterile filtration of the mixture, followed by
d) Lyophilisation.

The subject matter of the present invention is therefore the product of the above-described method of manufacture.

Furthermore, it is a preferred embodiment of the present invention that the method of preparation can be carried out between 4° C. and 37° C., or especially entirely at 4° C., thus facilitating the maintenance of tertiary structure of proteins and antigens, and also the activity of enzymes. The production of said carrier system complexes at such low temperatures leads to the benefits of maintaining native protein structure and surprisingly excellent storage stability of the biological agents. Preparation of an organosilicon carrier system under these conditions has not been described in any relevant literature to date. An unexpected advantage of mixing at a low temperature is the strength and stability of carrier system complex formation, whereby proteins form stable complexes with organosilicon molecules that maintain protein activity, as demonstrated in the examples below.

The claimed method generally requires a mixing time of 20-90 minutes, and in a preferred embodiment 20 or 30 minutes. This represents a significant reduction in time compared to the production of vesicles using organosilicon compounds when encapsulating any given biological agent, which usually requires 3-4 hours. Such a brief mixing step represents the satisfaction of a long-desired improvement in carrier system formation technology, whereby long mixture times are no longer required for stable complex formation between biological agents and organosilicon molecules.

A preferred embodiment of the present invention is that the method for preparation of the carrier system includes one or more of the following additives, including detergent solution, adjuvant solution, buffer solution, salt solution, non-cationic lipids, PEG and/or PEG-conjugates, lyosomes and/or other vesicles.

It is a preferred embodiment of the present invention that the carrier system is prepared by mixing the organosilicon compounds and additional biological agents in a buffer solution containing TRIS, HEPES, MOPS, MES or other commonly used biological buffer. The use of such biological buffer surprisingly enhanced the efficiency and strength of protein-organosilicon complex formation, thus providing a more stable carrier system.

The organosilicon, sugar organosilicon, amino sugar organosilicon compounds, its derivatives and/or salts or vesicles thereof according to the general formula 1 are intended to be used unsolved, in water, in aqueous or organic solvent or in a solvent mixture.

The biological agents, antigens, pre-antigens, antigen conjugates, antibodies, antibody conjugates, allergens, allergen extracts, nucleic acids, plasmids, proteins, peptides, pharmaceutical agents, immunologically active substances and or cosmetics are intended to be used unsolved, in water, in aqueous or organic solvent or in a solvent mixture.

The invention also has the task of providing methods for the production of emulsions based on non-phospholipid compounds which are chemically stable, can be prepared with a defined composition, can be prepared using a method that does not disrupt protein tertiary structure, and are unaffected by factors such as high temperature and exposure to light.

The method of the present invention requires no heating of the mixture above 37° C., thus distinguishing itself from prior art. The method of manufacture can be carried out at 4° C., or in any temperature range between 4° C. and 37° C. Furthermore, no dialysis is required to remove unbound or un-encapsulated compounds, thus providing a carrier system complex of precise composition that can be easily prepared.

Sizing of the carrier system complexes may be conducted in order to achieve a desired size range and relatively narrow distribution of particle sizes. Several techniques are available for the sizing of particles to a desired size. One sizing method, used for Liposomes and equally applicable to the present invention is described in U.S. Pat. No. 4,737,323, incorporated herein by reference.

Surprisingly, sonicating a carrier system suspension either by bath or probe sonication produces a progressive size reduction down to particles of less than about 50 nm in size. Homogenisation is also a method which relies on shearing energy to reduce particle size. In a typical homogenisation procedure, particles are re-circulated through a standard emulsion homogeniser until the selected particle sizes, typically between about 60 and 80 nm, are observed. In both methods the particle size distribution can be monitored by continual laser beam particle size discrimination. The sizing of the carrier system mixtures according to the method of manufacture results in unexpected reproducibility of carrier system formation, thus increasing the efficiency and reliability of the production method.

Extrusion of the particles through a small pore polycarbonate membrane or an asymmetric ceramic membrane is also an effective method for reducing the particle size to a relatively well defined size distribution. Typically, the suspension is cycled through the membrane one or more times until the desired particle size distribution is achieved. The particles may be extruded though successively smaller pore membranes, to achieve a gradual reduction in size. This provides the advantage of a regulated size reduction scheme allowing a precise tailoring of the final carrier system complex properties.

Lyophilisation is generally carried out using freeze drying technology controlling all aspects of the lyophilisation cycle. The shelf temperature ranges from −70° C. to 60° C., process condenser temperature as low as −85° C. and vacuum indication of 760 torr to 1 militorr. Dehydrated carrier system complexes are prepared by drying the preparations under reduced pressure either with or without one or more protective sugars, e.g. disaccharides such as trehalose and sucrose. Sugar organosilicon or amino sugar organosilicon compounds could be used as protective compounds for dehydration and/or lyophilisation of the carrier systems.

Such sizing and subsequent lyophilisation of the carrier system complexes leads to the unexpected advantage of greater storage stability and faster rehydration before administration. Carrier complexes are stable for extended periods of time at 4-8° C., thus facilitating the maintenance of native protein structure during storage.

Classical prophylactic vaccines provoke a humoral immune response, but have often been associated with an unfavourable safety profile. Moreover the development of classical vaccines has concentrated only on infectious diseases. A new generation of prophylactic and therapeutic vaccines is needed, including vaccines capable of inducing a stronger specific immune response than the classical preparation, vaccines possessing a more favourable safety profile than the classical preparation, and vaccines stable at high as well as low temperatures. Stability at 25-40° C. would be a great advantage in tropical territories. Furthermore, high reproducibility of the production process, with low inter- and intra-batch variability is required in order to avoid unexpected side effects due to the variability of potency.

Therefore, it is a preferred embodiment of the invention that the carrier system be used as a constituent in vaccines for therapeutic or prophylactic vaccination in humans and higher animals. The carrier system of the present invention can be used in the production of pharmaceutical agents used as vaccines for the prevention of infectious, chronic and life threatening diseases, or used directly as a vaccine for the prevention of infectious, chronic and life threatening diseases.

A further example of antigen administration is that of immunotherapy. Immunotherapy (or hypersensitisation) with allergen extracts was introduced in 1911 by Noon and Freeman. The treatment requires regular injections of allergen over a period of months. It is an established treatment for seasonal hay fever and for anaphylactic sensitivity to bees, wasps and hornets. In addition, immunotherapy is an effective treatment for selected cases of other allergic diseases including asthma.

Therefore, it is a preferred embodiment of the invention that the carrier system be used as a constituent in an immunotherapeutic for the desensitization of allergies in humans and higher animals, the use of the carrier system in the preparation of immunotherapeutics, or directly as immunotherapeutics.

Dust mites have been one of the major causes of allergy and asthma in the world. To date, more than 20 groups of allergenic proteins have been identified and characterized from dust mites. The group 13 allergen belongs to the fatty acid binding protein (FABPs) family. They are small cytosolic proteins that facilitate the transport and solubility of fatty acids. In humans they are highly tissue specific and have been characterized from at least eight different tissues.

Therefore it is a preferred embodiment of the present invention that the carrier system contains one or more house dust mite allergens. Furthermore, such a carrier system is intended for use as an immunotherapeutic and/or vaccine for the prevention and/or treatment of allergic disorders caused by house dust mite allergens, including anaphylaxis, seasonal hay fever, atopic dermatitis and allergic asthma. The preferred house dust mite allergens of the present invention are those listed in Table 1.

The combination of antigens with organosilicon molecules presents an unexpected increase in antigen stability, temperature resistance, enhanced bio-distribution, and combines multiple antigens in defined amounts with low intra-batch variability.

Surprisingly the carrier systems of the present invention comprising vaccines can induce long lasting cellular and humoral activity in experimental animals and long term safety (depot effect) of the administered vaccines and immunotherapeutics.

TABLE 1
List of house dust mite allergens of the present invention

| | | | MW (SDS- |
|---|---|---|---|
| No. | Allergen | Biochemical name | PAGE) |
| 1 | Der f 1 | Cysteine protease | 27 |
| 2 | Der f 2 | NPC2 family | 15 |
| 3 | Der f 3 | Trypsin | 29 |
| 4 | Der f 6 | Chymotrypsin | 25 |
| 5 | Der f 7 | — | 30-31 |
| 6 | Der f 10 | Tropomyosin | 37 |
| 7 | Der f 11 | Paramyosin | 98 |
| 8 | Der f 13 | Fatty acid binding protein | — |
| 9 | Der f 14 | Apolipophorin | 177 |
| 10 | Der f 15 | Chitinase | 98/109 |
| 11 | Der f 16 | Gelsolin/Vilin | 53 |
| 12 | Der f 17 | Calcium binding protein | 53 |
| 13 | Der f 18 | Chitinase | 60 |
| 14 | Der f 22 | — | — |

Monoclonal antibodies have been used to treat cancer by binding to specific proteins which are found on the surface of human cells and play a role in cell growth regulation. The epidermal growth factor receptor (EGFR) is one such target. EGFR exists on the cell surface and is activated by binding of its specific ligands. Upon activation, EGFR undergoes a transition from an inactive monomeric form to an active homodimer. EGFR dimerization stimulates the initiation of several signal transduction cascades leading to DNA synthesis and cell proliferation. Mutations that lead to EGFR over-expression or over-activity have been associated with a number of cancers. Mutations involving EGFR could lead to its constant activation which could result in uncontrolled cell division. Monoclonal antibodies have been developed as anti-cancer agents that can block the EGFR binding site, thus rendering it inactive and incapable of stimulating cell division.

Antibodies can be used either alone to kill cancer cells, or as carriers of other substances used also for treatment or for diagnostic purposes. For example, chemotherapeutic agents can be attached to monoclonal antibodies to deliver high concentrations of these toxic substances directly to the tumour cells. In theory, this approach is less toxic and more effective than conventional chemotherapy because it reduces the delivery of harmful agents to normal tissues. During the diagnostic process, monoclonal antibodies may be used to carry radioactive substances to cancer cells within the body, thus pinpointing the location of metastases that were previously undetected by other methods.

Therefore, it is a preferred embodiment of the invention that the carrier system be used in cancer diagnostics. A further preferred embodiment is that the carrier system of the present invention contains an antibody directed against EGFR or an EGFR antibody conjugate. Such carrier systems containing EGFR antibodies are intended to be used as a targeting system for biological agents in cancer diagnostics, or for the delivery of anti-tumour pharmaceutical medicaments. Another preferred embodiment is the use of carrier systems as a component in pharmaceutical compositions for the treatment of cancer in humans and higher animals, or to be used directly as anti-cancer agents. The organosilicon compounds of the present invention form surprisingly strong and stable complexes with antibodies. Carrier systems of the present invention containing antibodies demonstrate unexpected reductions in many of the common side effects associated with antibody treatments.

The formation of carrier systems with plasmids and nucleic acids is a further preferred embodiment of the present invention. Gene therapy relies on the transfer of nucleic acid molecules, usually in the form of expression cassettes, into target cells and particularly into the nucleus of a target cell. In order to bypass the lipid membrane of the cell, lipid-like molecules allow masking of the nucleic acid material to resemble a membrane and thus facilitate uptake of the gene therapeutic nucleic acid. Similarly, nucleic acids can be used to reduce or eliminate gene expression in any given cell through antisense technology, and the use of carrier system complexes containing silencing nucleic acid molecules is also a preferred embodiment of the present invention. The transportation of nucleic acids to cells in vivo represents a crucial step in the development of gene therapy technology, due to the many problems associated with efficient cell targeting and transfection. The use of carrier molecules with nucleic acids, as described in the present invention, represents a significant step forward in the efficiency of preparing therapeutic gene vehicles.

Therefore it is a preferred embodiment of the invention that the carrier system be used as a carrier for nucleic acids.

Cationic lipids, glycolipids, phospholipids, cholesterol or derivatives thereof, and equivalent molecules known to those of skill in the art, can also be included in the carrier systems of the present invention. Cationic lipids can comprise preferably DOTMA (N-[1′-(2,3-dioleyoxy)propyl]-N,N,N-trimethylammonium-chloride), DODAC (N,N-dioleyl-N,N-dimethylammoniumchloride), DDAB (didodecyldimethylammonium bromide) and stearylamine and other aliphatic amines and the like. Carrier systems containing different types of polar (positively or negatively charged) and neutral (electrochemically neutral) lipids represent another preferred embodiment of the invention, examples of such lipids including phosphatidyl ethanolamine, phosphatidyl Inositol, phosphatidyl glycine, phosphatidyl glycerol, lipofectamine, cholesterol.

Therefore, in a preferred embodiment of the present invention carrier systems contain one or more positively or negatively charged polar lipids, electrochemically neutral lipids, glycolipids, phospholipids, cholesterol or derivatives thereof. The inclusion of lipids in the organosilicon carrier systems led to surprising enhanced complex formation strength.

Polyethylene glycol (PEG) is a polymer of ethylene oxide of varying molecular weights. The liquid PEGs have an average molecular weight of 200-600 daltons (PEG 200-PEG 600). To couple PEG to lipids in order to form PEG-lipid conjugates, the PEG (generally mono-methoxy PEG) is first activated. Several methods can be used to achieve this activation and coupling. PEG could be covalently attached to the lipids using the two terminal hydroxyl groups. Surprisingly, when attached to various protein medications or other biological agents of the present invention, PEG allowed a slowed clearance of the carried protein or molecule from the blood. This makes for a longer acting medicinal effect and reduces toxicity, and it allows longer dosing intervals. PEG can also be conjugated to phospholipids, which are then used in liposome formation for drug delivery. Furthermore, PEG can be conjugated to ceramides, and included in liposome vesicle preparations. These ceramide-PEG conjugates further extend the circulation and release time of the vesicle-associated biological agent.

It was a surprising advantage that the carrier system complexes containing PEG-conjugated organosilicon, sugar organosilicon, amino sugar organosilicon compounds, showed good physical stability on storage of the carrier system. Furthermore, when free liquid PEG was used with the organosilicon, sugar organosilicon, and/or amino sugar organosilicon compounds with the proteins according to the preparation process of this invention, the aqueous solubility and dissolution characteristics of poorly soluble proteins and compounds in the mixture were unexpectedly enhanced. In further embodiments of the present invention, the PEG can be covalently attached to the different types of polar (positively or negatively charged) and neutral (electrochemically neutral) lipids, including but not limited to phosphatidyl ethanolamine, phosphatidyl inositol, phosphatidyl glycine, phosphatidyl glycerol, lipofectamine, cholesterol.

Therefore, it is a preferred embodiment of the invention that the carrier system contains free liquid PEG, PEG conjugates and/or PEG-ceramide conjugates. Such conjugates can contain PEG covalently attached to the organosilicon, sugar organosilicon, amino sugar organosilicon compounds, the biological agents and/or lipids.

Furthermore, in a preferred embodiment the method for preparing the carrier system incorporates non-cationic lipids as PEG-lipid conjugates, whereby said PEG-lipid conjugate can be PEG-ceramide conjugates.

The carrier systems of the present invention can be used as carriers or targeting systems for the administration of a large range of biologically active agents, such as pharmaceutical agents or drugs. The properties of the carrier systems of the present invention lead to beneficial aspects in production, stability, delivery, dose and administration of drugs used to treat human or animal disease.

Therefore, in a preferred embodiment of the invention the carrier system is used in the production of a pharmaceutical agent to be used for the treatment of infectious, acute, chronic and life threatening diseases, as directly as a pharmaceutical agent for the treatment of infectious, acute, chronic and life threatening diseases.

Specific targeting of the carrier systems is also an intended objective of the present invention. Antibodies may be used to bind to the organosilicon molecules and to direct the carrier system and its biological agent to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing carrier systems to particular cell types.

Siosomes and organosilicon compounds have become increasingly important as a vehicle for the controlled delivery of cosmetics. Liposomes and siosomes can encapsulate many types of cosmetic agents. Such encapsulation provides improved uptake, adhesion, and persistence of active ingredients in skin and hair products. However, such encapsulations suffer from the same disadvantages as described above, and rely solely on vesicle formation.

Therefore, it is a preferred embodiment of the invention that the carrier system be used in the preparation of cosmetics, or directly as cosmetic.

Furthermore, the subject matter of this invention is to make available kits for the preparation of carrier systems according to the present invention. The organosilicon, sugar organosilicon, and/or amino sugar organosilicon compounds are stored in a separate container to the biological agents within the kit. Also included is information on how to use each part of the kit. By keeping the active substances, adjuvants, and/or additives used in the production of the carrier system separate, it is possible to prepare the combinations in different concentrations and use and/or administer the different carrier system variants, produced via the kit, in different schemes (for example simultaneously, sequentially or repeated).

Therefore, an object of the present invention is to provide a kit for the preparation of a carrier system according to the present invention, whereby the silicon organic compound, sugar organosilicon and/or amino sugar organosilicon compounds, its derivatives, and/or vesicles prepared from them are stored in a separate container from the biological agents, antigens, antigen conjugates, nucleic acids, plasmids, proteins, peptides, allergens, allergen extracts, pharmaceutical agents, immunologically active substances, cosmetics, additives, and/or adjuvants. Solvents for reconstitution such as sterile water, detergent buffer or oil, are also stored separately, allowing the administration of different concentrations and combinations of various agents.

The invention provides carrier systems comprising organosilicon, sugar organosilicon, amino sugar organosilicon compounds, and/or its siosome vesicles, with pharmaceutical agents, immunologically active substances and/or biologically active substances, and a method for their production, with many advantages, for example: no unbound material and substance will be washed away during production (as is the case during the preparation of liposomes and other vesicle technologies) resulting in very high combination production yield, usually more than 90%, the carrier systems of the present invention demonstrate efficient production costs due to simple production methods and procedures and there is a well-defined content of the antigens, pharmaceutical agents and/or biologically active substances within the carrier system.

Furthermore and surprisingly the carrier systems according to the invention show the following advantages: they induce the correct/desired type of immunity when administered with antigens as vaccines or immunotherapeutics, carrier systems are stable on storage (this is particularly important for living vaccines which are normally required to be kept cold, for example a complete ‘cold chain’ from manufacturer to clinic, which is by no means easy to maintain), they demonstrate sufficient immunogenicity, carrier systems maintain the tertiary and three dimensional structure of the biological agents and are highly reproducible. Furthermore, the carrier systems of the present invention, and their method of manufacture, represent a significant development of technology where the prior art had pursued efforts in a different direction. The method of manufacture does not rely on the slow and comparatively harsh conditions of vesicle encapsulation, and represents a change of direction in carrier system formation.

### DEFINITIONS AND PREFERRED EMBODIMENTS OF THE INVENTION

The following terms are defined as follows.

“Silane” is a chemical compound containing silicone, particularly with the chemical formula SiH₄, although other silicon-containing compounds may also be referred to herewith.

“Organosilicons” are organic compounds containing carbon-silicon bonds (C—Si).

“Sugar organosilicons” are organic compounds containing carbon-silicon bonds (C—Si) and at least one sugar group.

“Amino sugar organosilicons” are organic compounds containing carbon-silicon bonds (C—Si), at least one sugar group and one amino acid group. Examples of amino sugars and derivatives are well known in the art as glucosamine, galactosamine, mannosamine, neuramine acid, muramine acid, N-acetylglucosamine, further examples include acylated sugars and aminosugars. Amino sugar organosilicons comprise residues such as glucosamine, N-acetyl glucosamine, sialic acid or galactosamine, where the amino sugar residues can be the same or different, and represent the removal of a hydrogen from monoaminosaccharide and/or diaminosaccharide and/or polysaccharide.

“Siloxanes” are a class of organic or inorganic chemical compounds of silicon, oxygen, and usually carbon and hydrogen, based on the structural unit R₂SiO, where R is an alkyl group, usually methyl.

“Siosomes” are the vesicles created from organosilicon compounds. Siosomes consist of at least one concentric, self-contained layer of organosilicon compounds with the organosilicon general structure. An aqueous compartment is enclosed by the bimolecular organosilicon membrane.

“Sugar-siosomes” refer to siosomes consisting of sugar organosilicon compounds.

As referred to in this patent “blank siosomes” refers to any and all vesicles prepared from organosilicon compounds without encapsulated and/or entrapped pharmacologically and/or immunologically active agents. Specific examples of “blank siosomes” are siosomes filled with water, salts or buffer.

“Liposomes” are the vesicles created from phospholipids. Liposomes consist of at least one concentric, self-contained layer of phospholipids and an aqueous compartment enclosed by the bimolecular phospholipid membrane.

“Encapsulations” are to be understood as the capture by a siosome or liposome of a dissolved hydrophilic solute within the region of aqueous solution inside a hydrophobic membrane, whereby the dissolved hydrophilic solutes cannot readily pass through the lipid bi-layer.

“Entrapments” are to be understood as the capture by a liposome or siosome of hydrophobic solutes dissolved in the hydrophobic region of the bi-layer membrane.

“Combinations” or “complex combinations” of the present invention are intended to be understood as complexes formed between the organosilicon, sugar organosilicon, amino sugar organosilicon compounds, their derivatives, salts and/or the vesicles formed from them, with the biological agents, selected from antigens, pre-antigens, antigen conjugates, antibodies, pre-antibodies, antibody conjugates, allergens, allergen extracts, nucleic acids, plasmids, proteins, peptides, pharmaceutical agents, immunologically active substances and/or cosmetics, whereby the complexes are held together via the sum force of non-covalent bonds including Hydrophobic interactions between non-polar regions, such as the hydrophobic fatty acid chains of the organosilicon, sugar organosilicon, amino sugar organosilicon compounds and siosomes, non-specific protein-protein interactions between protein segments of an allergen and/or the peptide residues of the organosilicon, sugar organosilicon, amino sugar organosilicon compounds, protein-saccharide interactions, Van der Waals interactions including, dipole-dipole interactions, dipole-induced dipole interactions, dispersion forces (London forces), electrostatic interactions between charged resides, for example peptide residues and organosilicon, sugar organosilicon, amino sugar organosilicon compounds, and salts, ion-dipole forces; for example those between the salts of the buffer used in the preparation of the complex and H₂O, and ion-ion forces; for example those involving the salts of the buffer solution.

An “antigen” is to be understood as a substance that prompts the generation of antibodies and can cause an immune response.

A “pre-antigen” is to be understood as an antigen in an inactive state prior to processing to the active form.

An “antigen conjugate” is to be understood as an antigen conjugated covalently to another biological agent, such as an enzyme or other protein, or organosilicon molecule.

A “pre-antibody” is to be understood as an antibody in an inactive state prior to processing to the active form.

An “antibody conjugate” is to be understood as an antibody conjugated covalently to another biological agent, such as an enzyme or other protein, or organosilicon molecule.

The term “allergen” refers to a substance, protein or non-protein, capable of inducing allergy or specific hypersensitivity or an extract of any substance known to cause allergy. Almost any substance in the environment can be an allergen. The list of known allergens includes plant pollens, spores of mold, food preservatives, dyes, drugs, inorganic chemicals and vaccines. Allergens can enter the body by being inhaled, swallowed, touched or injected. Following primary exposure to an allergen, subsequent exposures result in hypersensitivity (allergic) reactions which may be immediate or delayed, local or systemic and include anaphylaxis and contact dermatitis.

The term “allergen extract” refers to a natural extract of multiple allergens, protein or non-protein, capable of inducing allergy or specific hypersensitivity or an extract of any substance known to cause allergy.

A “pharmaceutical agent” is to be understood as any medicament, intended for use in the diagnosis, cure, mitigation, treatment, or prevention of disease in humans or animals.

An “immunologically active substance” is to be understood as any substance that leads to an immune response in a human or animal patient.

As used herein “sugar” includes any and all monosaccharide, disaccharide, polysaccharide, amino-sugar or hydroxyl carbon acid, it's derivatives, salts and/or residues remaining after the removal of a hydrogen atom from it. The following are suitable examples of suitable monosaccharides; pentoses such as arabinose, ribose and xylose as well as hexoses such as glucose, mannose, galactose and fructose. Suitable amino sugars include e.g. glucosamin and galactosamin. A suitable carbon acid for example is glucronic acid. The hydroxyl carbon acids' hydroxyl groups can be free, partially derivatized or fully derivatized (protective groups) specific examples of amino sugar silicon compounds are listed herein.

A “patient” for the purposes of the present invention includes both humans and other animals, particularly mammals, and other organisms. Thus the methods are applicable to both human therapy, vaccinations, and veterinary applications. In the preferred embodiment the patient is a mammal, the most preferred being a human.

The term “animal” refers to an organism with a closed circulatory system of blood vessels and includes birds, mammals and crocodiles. The term “animal” used here also includes human subjects.

An “immunologically effective amount” is the quantity of a compound, composition or carrier system of the present invention which is effective in yielding the desired immunologic response.

The terms “treating cancer,” “therapy,” and the like refer generally to any improvement in the mammal having the cancer wherein the improvement can be ascribed to treatment with the compounds of the present invention. The improvement can be either subjective or objective. For example, if the mammal is human, the patient may note improved vigour or vitality or decreased pain as subjective symptoms of improvement or response to therapy. Alternatively, the clinician may notice a decrease in tumour size or tumour burden based on physical exam, laboratory parameters, tumour markers or radiographic findings. Some laboratory obtained results which the clinician may observe to check for any response to therapy include normalization of tests such as white blood cell count, red blood cell count, platelet count, erythrocyte sedimentation rate, and various enzyme levels. Additionally, the clinician may observe a decrease in a detectable tumour marker(s). Alternatively, other tests can be used to evaluate objective improvement such as sonograms, nuclear magnetic resonance testing and positron emissions testing.

“Inhibiting the growth of tumour cells” can be evaluated by any accepted method of measuring whether growth of the tumour cells has been slowed or diminished. This includes direct observation and indirect evaluation such as subjective symptoms or objective signs as discussed above.

Accordingly, the carrier systems of the invention are administered to cells, tissues, healthy volunteers and subjects and/or patients. Herein what is meant by “administered” is the administration of a therapeutically effective dose of the candidate agents of the invention to a cell either in cell culture or in a patient. Herein what is meant by “therapeutically effective dose” is a dose that produces the effects for which it is administered. The exact dose will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. Herein what is meant by “cells” is almost any cell in which mitosis or miosis can be altered.

Additional “carriers” may be used in the “carrier system” of the present invention, and include any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the carrier systems.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

The term “pharmaceutically acceptable salt” refers to those salts of compounds which retain the biological effectiveness and properties of the free bases and which are obtained by reaction with inorganic acids such as hydrochloric acid, hydrobromic acid, sulphuric acid, nitric acid, phosphoric acid, methanesulphonic acid, ethanesulphonic acid, toluenesulphonic acid, salicylic acid and the like. Pharmaceutically acceptable salts include alkali metal salts, such as sodium and potassium, alkaline earth salts and ammonium salts.

Therefore, as used herein, “cancer” refers to all types of cancer or neoplasm or malignant tumours found in mammals, including carcinomas and sarcomas. Examples of cancers are cancer of the brain, breast, cervix, colon, head & neck, kidney, lung, non-small cell lung, melanoma, mesothelioma, ovary, sarcoma, stomach, uterus and Medulloblastoma.

The present invention relates to the use of organosilicon, sugar organosilicon, amino sugar organosilicon compounds, their derivatives, salts and/or the vesicles formed from them in a carrier system with antigens, pre-antigens, antigen conjugates, antibodies, antibody conjugates, allergens, allergen extracts, nucleic acids, plasmids, proteins, peptides, pharmaceutical agents, immunologically active substances and/or cosmetics, for the manufacturing of a pharmaceutical, immunological and/or cosmetic composition for the different indications.

The pharmaceutical composition of the invention optionally comprises of one or more pharmaceutically acceptable adjuvants, excipients, carriers, buffers, diluents and/or customary pharmaceutical auxiliary substances. The composition of the invention is administered in a pharmaceutically acceptable formulation. The present invention pertains to any pharmaceutically acceptable formulations, such as synthetic or natural polymers in the form of macromolecular complexes, nanocapsules, microspheres, or beads, and lipid-based formulations including oil-in-water emulsions, micelles, mixed micelles, synthetic membrane vesicles, and resealed erythrocytes. In addition to the said composition and the pharmaceutically acceptable polymer, the pharmaceutically acceptable formulation of the invention can comprise additional pharmaceutically acceptable carriers and/or excipients. As used herein, a pharmaceutically acceptable carrier includes any and all solvents, dispersion media, coatings, antibacterial and anti fungal agents, isotonic and absorption delaying agents, and the like that are physiologically compatible. For example, the carrier can be suitable for injection into the cell, tissues, organs and/or blood. Excipients include pharmaceutically acceptable stabilizers and disintegrants. In another embodiment, the pharmaceutically acceptable formulations comprise lipid-based formulations. Any of the known lipid-based drug delivery systems can be used in the practice of the invention. For instance, multivesicular liposomes (MVL), multilamellar liposomes (also known as multilamellar vesicles or MLV), unilamellar liposomes, including small unilamellar liposomes (also known as unilamellar vesicles or SUV), large unilamellar liposomes (also known as large unilamellar vesicles or LUV), multivesicular siosomes (MVS), multilamellar siosomes (MLS), unilamellar siosomes including small unilamellar siosomes can all be used so long as a sustained release rate of the carrier system composition of the invention can be established.

In one embodiment, the lipid-based formulation can be a multivesicular liposome system. Other phospholipids or other lipids may also be used. Examples of lipids useful in synthetic membrane vesicle production include phosphatidylglycerols, phosphatidylcholines, phosphate-idylserines, phosphatidyl-ethanolaminos, sphingolipids, cerebrosides, and gangliosides. Preferably phospholipids including egg phosphatidylcholine, dipalmitoylphosphat-idylcholine, distearoylphosphatidylcholine, dioleoylphos-phatidylcholine, dipalmitoylphosphatidylglycerol, and dioleoylphosphatidyl-glycerol are used. In another embodiment, the composition containing the carrier system of the invention may be incorporated or impregnated into a bioabsorbable matrix. In addition, the matrix may be comprised of a biopolymer. A suitable biopolymer for the present invention can include also one or more macromolecules selected from the group consisting of collagen, elastin, fibronectin, vitronectin, laminin, polyglycolic acid, hyaluronic acid, chondroitin sulphate, dermatan sulphate, heparin sulphate, heparin, fibrin, cellulose, gelatine, polylysine, echinonectin, entactin, thrombospondin, uvomorulin, biglycan, decorin, and dextran. The formulation of these macromolecules into a biopolymer is well known in the art. In a preferred embodiment, the therapeutic composition is not immunogenic when administered to a human patient for therapeutic purposes.

The therapeutic carrier system of the present invention can include pharmaceutically acceptable salts of the components therein. Pharmaceutically acceptable salts include the acid addition salts that are formed with inorganic acids such as hydrochloric or phosphoric acids, or such organic acids as acetic, tartaric, mandelic and the like. Salts formed with the free carboxyl groups of the pharmaceuticals and/or additives and/or adjuvants derivatives can also be derived from inorganic bases such as sodium, potassium, ammonium, calcium or ferric hydroxides, and such organic bases as isopropylamine, trimethylamino, 2-ethylamino ethanol, histidine, procaine and the like. Physiologically tolerable carriers are well known in the art. Exemplary liquid carriers are sterile aqueous solutions which contain no materials in addition to the active ingredients and water, or contain a buffer such as sodium phosphate at a physiological pH value, in a physiological amount of saline or both, for example phosphate-buffered saline. Further still, aqueous carriers can contain more than one buffer salt, as well as salts such as sodium and potassium chlorides, dextrose, propylene glycol, polyethylene glycol and other solutes. Liquid compositions can also contain liquid phases in addition to and to the exclusion of water. Exemplary examples of such additional liquid phases include glycerine, vegetable oils such as cottonseed oil, organic esters such as ethyl oleate, and water-oil emulsions.

The pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatine or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874, to form osmotic therapeutic tablets for control release.

A pharmaceutical carrier system may also, or alternatively, contain one or more drugs, which may be linked to a modulating agent or may be free within the composition. Virtually any drug may be administered in combination with a modulating agent as described herein, for a variety of purposes as described below. Examples of types of drugs that may be administered with a modulating agent include analgesics, anaesthetics, antianginals, antifungals, antibiotics, anti-cancer drugs (e.g., taxol or mitomycin C), anti-inflammatories (e.g., ibuprofen and indomethacin), anthelmintics, antidepressants, antidotes, antiemetics, antihistamines, antihypertensives, antimalarials, antimicrotubule agents (e.g., colchicine or vinca alkaloids), antimigraine agents, antimicrobials, antiphsychotics, antipyretics, antiseptics, anti-signalling agents (e.g., protein kinase C inhibitors or inhibitors of intracellular calcium mobilization), antiarthritics, antithrombin agents, antituberculotics, antitussives, antivirals, appetite suppressants, cardioactive drugs, chemical dependency drugs, cathartics, chemotherapeutic agents, coronary, cerebral or peripheral vasodilators, contraceptive agents, depressants, diuretics, expectorants, growth factors, hormonal agents, hypnotics, immunosuppression agents, narcotic antagonists, parasympatho-mimetics, sedatives, stimulants, sympathomimetics, toxins (e.g., cholera toxin), tranquilizers and urinary anti-infectives.

Formulations for oral use may also be presented as hard gelatine capsules where in the active ingredient is mixed with an inert solid diluent, for example calcium carbonate, calcium phosphate or kaolin, or as soft gelatine capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate polyvinyl-pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified, for example sweetening, flavouring and colouring agents, may also be present.

The pharmaceutical carrier systems of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soya bean, lecithin, and esters/partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain demulcent, preservatives, flavouring agents and colouring agents. The pharmaceutical carrier systems may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally-acceptable diluent or solvent, for example as absolution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

For administration to patients, the active substances of the present invention are mixed with a pharmaceutically acceptable carrier or diluent in accordance with routine procedures. Therapeutic and/or immunologic formulations will be administered by intravenous infusion or by subcutaneous injection. The formulations can also contain, if desired, other therapeutic agents.

The invention relates also to a process or a method for the treatment of the abovementioned pathological conditions. The compounds of the present invention can be administered prophylactically or therapeutically, preferably in an amount that is effective against the mentioned disorders, to a warm-blooded animal, for example a human, requiring such treatment, the compounds preferably being used in the form of pharmaceutical carrier systems.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intra-muscular administration and formulation.

In certain applications, the pharmaceutical carrier systems disclosed herein may be delivered via oral administration. As such, these carrier systems may be formulated with an inert diluent or with an assimilable edible carrier, or they may be enclosed in hard- or soft-shell gelatine capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatine; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavouring agent, such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup or elixir may contain the active compound sucrose as a sweetening agent, methyl and propylparabens as preservatives, a dye and a flavouring, such as cherry or orange flavour. Of course, any material used in preparing any dosage unit form should be pharmaceutically pure and substantially non-toxic in the amounts employed. In addition, the active compounds may be incorporated into sustained-release preparation and formulations.

Typically, these formulations contain at least 0.1% of the active compound of the invention or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful carrier system may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the carrier systems of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerine and potassium bicarbonate, or dispersed in a dentifrice, or added in a therapeutically-effective amount to a carrier system that may include water, binders, abrasives, flavouring agents, foaming agents, and humectants. Alternatively the carrier systems may be fashioned into a tablet or solution form that may be placed under the tongue or otherwise dissolved in the mouth.

In certain circumstances it will be desirable to deliver the pharmaceutical carrier systems disclosed herein parenterally, intravenously, intramuscularly, subcutaneously or even intraperitoneally. Solutions of the active compounds as free bases or as pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable carrier systems can be brought about by the use in the carrier systems of agents delaying absorption, for example, aluminium monostearate and gelatine.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some necessary variation in the dosage will occur depending on the condition of the subject being treated. The person responsible for administration will, in any event, determine the appropriate dose for the individual subject. Moreover, for human administration, preparations should meet sterility, pyrogenicity, and the general safety and purity standards as required by national or regional offices of biologics standards.

Sterile injectable solutions are prepared by incorporating the active compounds in the required amount in the appropriate solvent with various ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The carrier systems disclosed herein may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

In certain embodiments, the pharmaceutical carrier systems may be delivered by intranasal sprays, inhalation, and/or other aerosol delivery vehicles. Likewise, the delivery of drugs using intranasal microparticle resins and lysophosphatidyl-glycerol compounds are also well-known in the pharmaceutical arts.

In certain embodiments, the inventors contemplate the use of nanocapsules, microparticles, microspheres, and the like, in the production of the carrier systems of the present invention. Such formulations may be preferred for the introduction of pharmaceutically-acceptable formulations of the carrier system or constructs disclosed herein.

The invention provides for pharmaceutically-acceptable nanocapsule formulations of the carrier systems of the present invention. Nanocapsules can generally entrap compounds in a stable and reproducible way. To avoid side effects due to intracellular polymeric overloading, such ultra fine particles (sized around 0.1.mu.m) should be designed using polymers able to be degraded in vivo. Biodegradable polyalkyl-cyanoacrylate nanoparticles that meet these requirements are contemplated for use in the present invention.

The subjects treated will typically comprise of mammals and will preferably be human subjects, e.g., human cancer subjects. The compounds of the invention may be used alone or in combination. Additionally, the treated compounds may be utilized with other types of treatments, e.g., cancer treatments. For example, the subject compounds may be used with other chemotherapies, e.g., tamoxifen, taxol, methothrexate, biologicals, such as antibodies, growth factors, lymphokines, or radiation, etc. Combination therapies may result in synergistic results. The preferred indication is cancer, especially the cancers identified previously.

The preferred organosilicon, sugar organosilicon, and amino sugar organosilicon compounds are listed in table 2.

TABLE 2
Preferred organosilicon, sugar organosilicon, and amino sugar
organosilicon compounds for carrier system production.

| | | Molecular | MW |
|---|---|---|---|
| No, | Structure and Name | Formula | (Da) |
| Sil 1 | 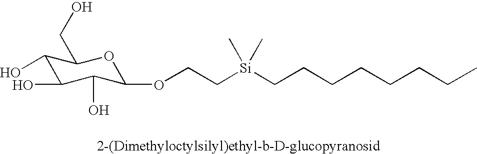 | C18H38O6Si | 378.59 |
| Sil 2 | 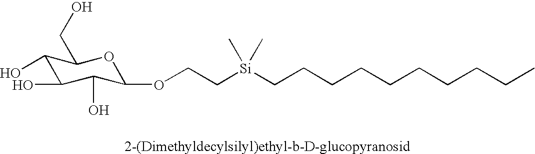 | C20H42O6Si | 406.64 |
| Sil 3 | 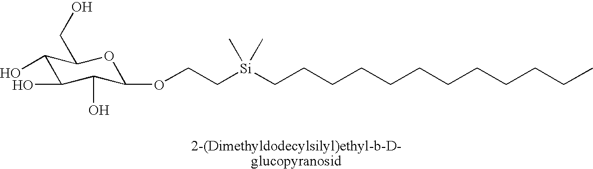 | C22H46O6Si | 434.69 |
| Sil 4 | 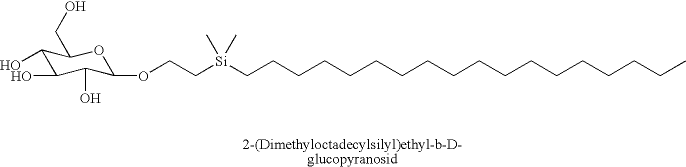 | C28H58O6Si | 518.86 |
| Sil 5 | 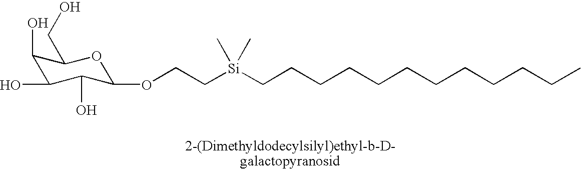 | C22H46O6Si | 434.69 |
| Sil 6 | 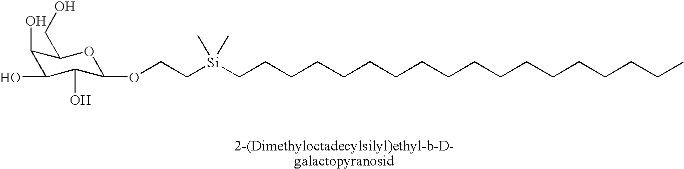 | C28H58O6Si | 518.86 |
| Sil 7 | 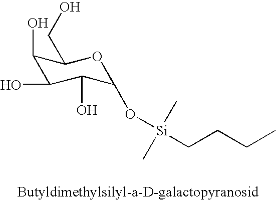 | C12H26O6Si | 294.42 |
| Sil 8 | 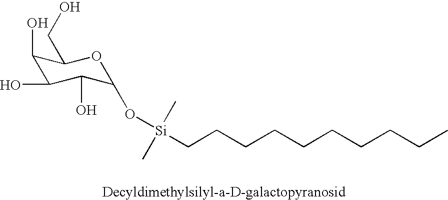 | C18H38O6Si | 378.59 |
| Sil 9 | 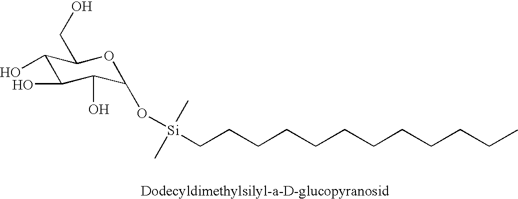 | C20H42O6Si | 406.64 |
| Sil 10 | 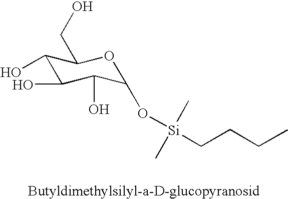 | C12H26O6Si | 294.42 |
| Sil 11 | 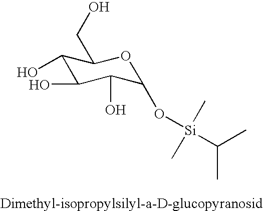 | C11H24O6Si | 280.40 |
| Sil 12 | 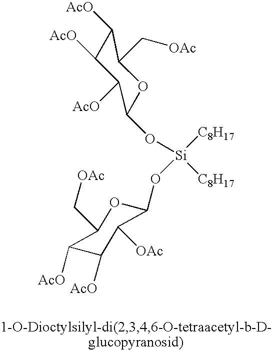 | C44H72O20Si | 949.14 |
| Sil 13 | 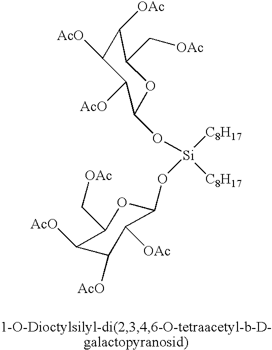 | C44H72O20Si | 949.14 |
| Sil 14 | 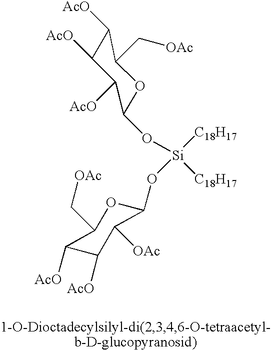 | C64H112O20Si | 1229.68 |
| Sil 15 | 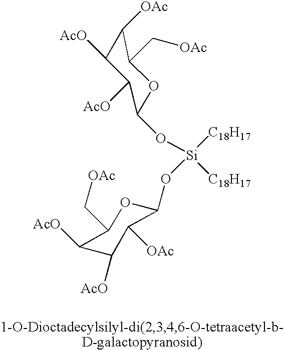 | C64H112O20Si | 1229.68 |
| Sil 16 | 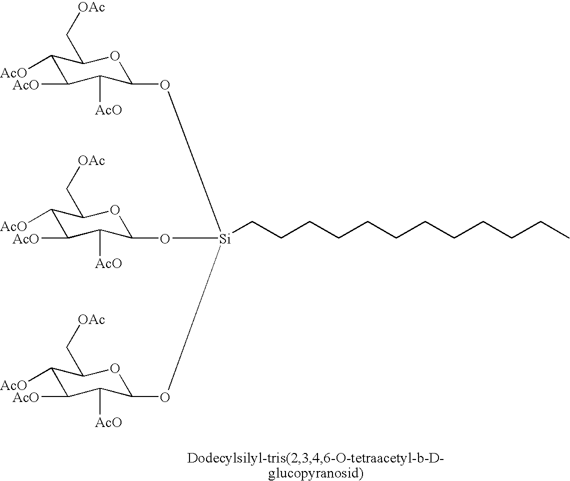 | C54H82O30Si | 1239.32 |
| Sil 17 | 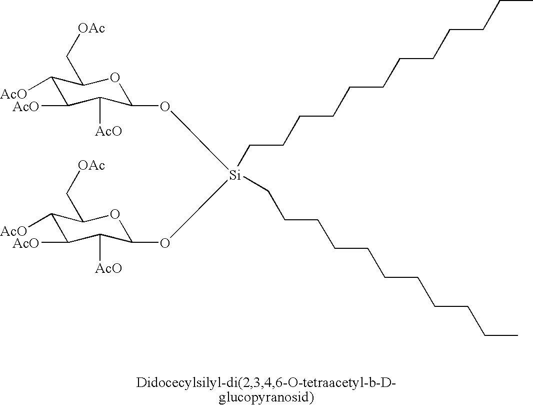 | C52H88O20Si | 1061.36 |
| Sil 18 | 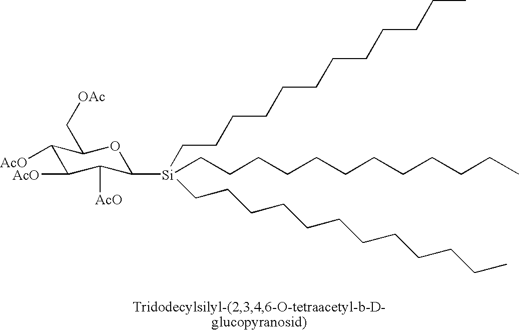 | C50H94O10Si | 883.39 |
| Sil 19 | 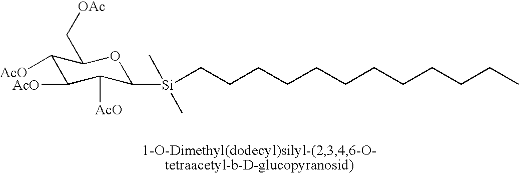 | C28H50O10Si | 574.79 |
| Sil 20 | 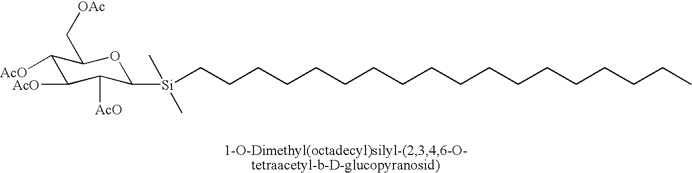 | C34H62O10Si | 658.95 |
| Sil 21 | 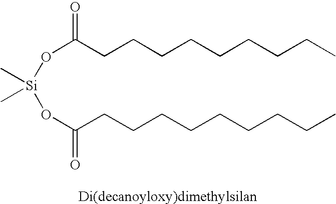 | C22H44O4Si | 400.68 |
| Sil 22 | 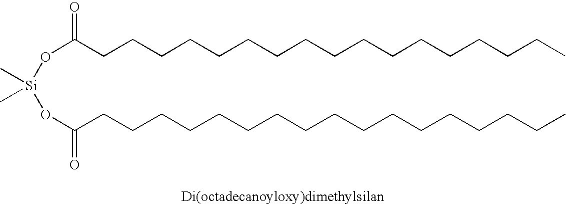 | C38H76O4Si | 625.11 |
| Sil 23 | 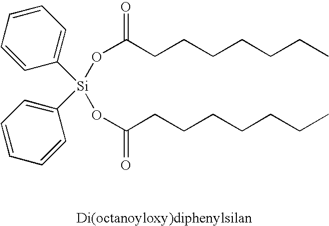 | C28H40O4Si | 468.71 |
| Sil 24 | 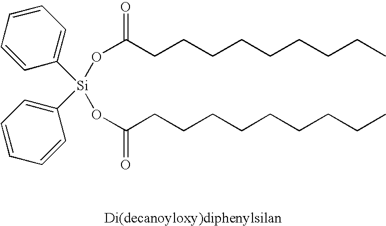 | C32H48O4Si | 524.82 |
| Sil 25 | 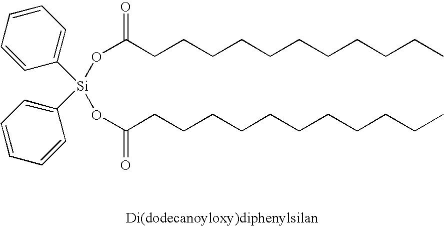 | C36H56O4Si | 580.93 |
| Sil 26 | 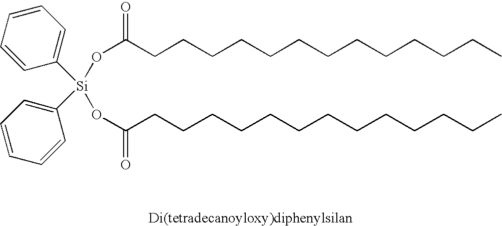 | C40H64O4Si | 637.04 |
| Sil 27 | 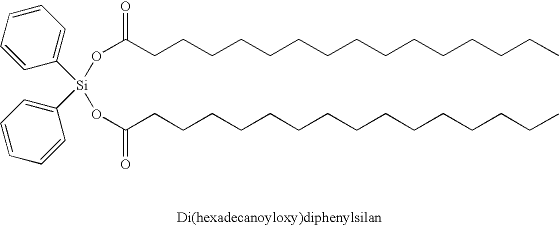 | C44H72O4Si | 693.15 |
| Sil 28 | 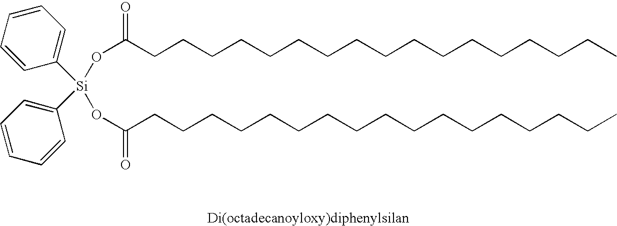 | C48H80O4Si | 749.26 |
| Sil 29 | 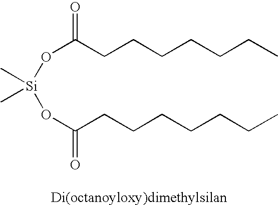 | C18H36O4Si | 344.57 |
| Sil 30 | 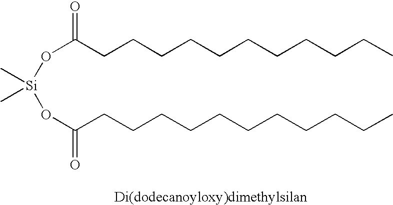 | C26H52O4Si | 456.79 |
| Sil 31 | 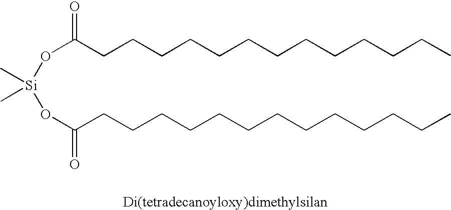 | C30H60O4Si | 512.90 |
| Sil 32 | 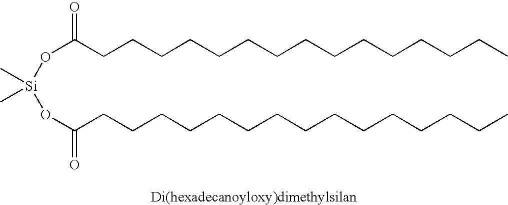 | C34H68O4Si | 569.00 |
| Sil 33 | 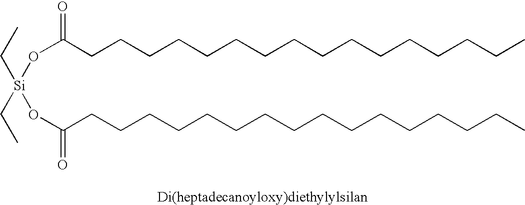 | C38H76O4Si | 625.11 |
| Sil 34 | 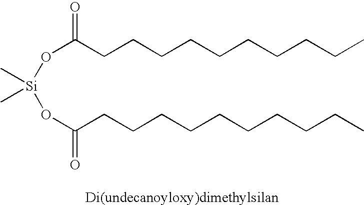 | C24H48O4Si | 428.73 |
| Sil 35 | 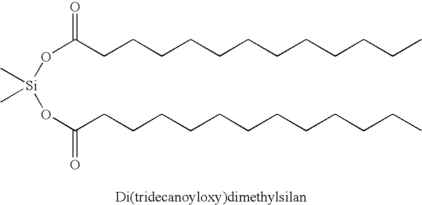 | C28H56O4Si | 484.84 |
| Sil 36 | 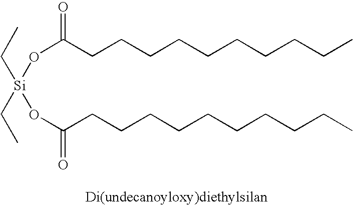 | C26H52O4Si | 456.79 |
| Sil 37 | 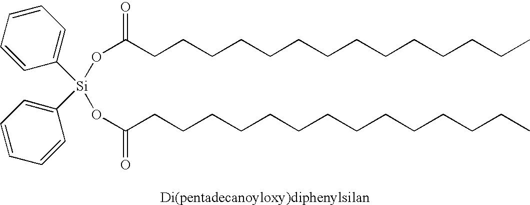 | C42H68O4Si | 665.09 |
| Sil 38 | 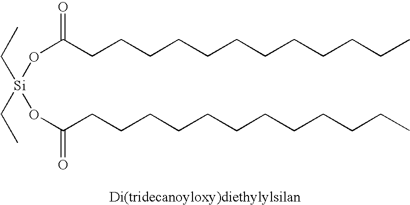 | C30H60O4Si | 512.90 |
| Sil 39 | 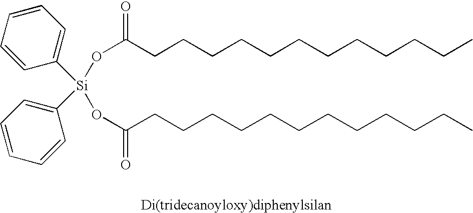 | C38H60O4Si | 608.99 |
| Sil 40 | 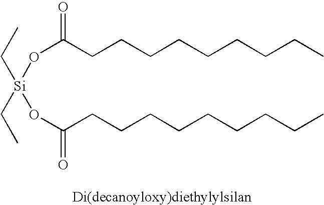 | C24H48O4Si | 428.73 |
| Sil 41 | 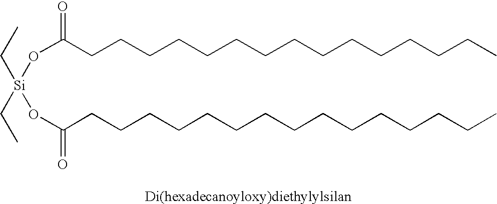 | C36H72O4Si | 597.06 |
| Sil 42 | 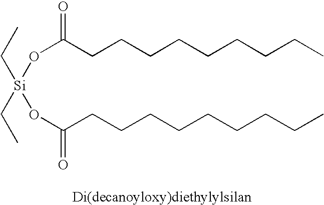 | C24H48O4Si | 428.73 |
| Sil 43 | 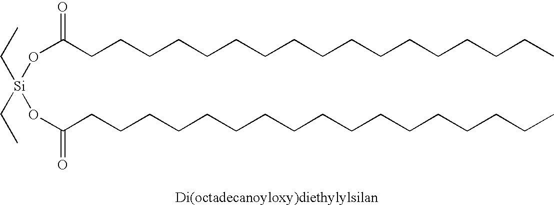 | C40H80O4Si | 653.17 |
| Sil 44 | 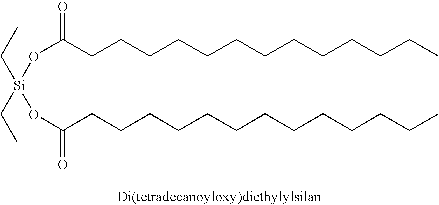 | C32H64O4Si | 540.95 |
| Sil 45 | 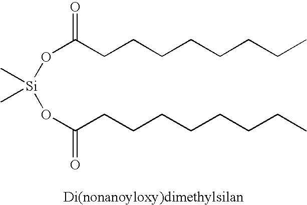 | C20H40O4Si | 372.63 |

### EXAMPLES

Unless otherwise defined, all technical and scientific terms used herein have the same meaning as commonly understood by a person of ordinary skill in the art to which this invention belongs. Although methods and materials similar or equivalent to those described herein can be used in the practice or testing of the present invention, suitable methods and materials are described below.

Determining Efficiency of Carrier System Protein Complex Formation with the Organosilicon, Sugar Organosilicon, Amino Sugar Organosilicon Compounds and/or Blank (Unloaded) Vesicles Thereof.

Carrier system formation efficiency, or protein complex formation between the organosilicon, sugar organosilicon, and/or amino sugar organosilicon compounds with biological protein agents, was calculated after centrifugation by quantification of the amount of the protein in the protein-complex fraction and the free non-complexed proteins presents in the aqueous supernatant phase. The protein was quantified using a modified Lowry method, Peterson G. L. 1983 “Determination of total protein”, Methods Enzymology 91: 95-119.

### In-Vivo Protocol for the Immunogenicity Tests

Adult, pathogen-free New Zealand white rabbits were used in the immunogenicity tests. The rabbits were grouped (n=4) and immunized with the antigen and/or antigen mixture. Two sets of injections per rabbit on day 0 and 14. Intramuscular (i.m) or subcutaneous injections (s.c) were given in the hind legs following a standard protocol, with the first injection set given in the right leg, the second injection set given in the left leg. The injection sites were labelled for later identification. To assess immunogenicity, sera were prepared from blood samples obtained from each rabbit on day 0 (control) and day 19 (5 days after the second injection). Blood (15 ml per bleed) was collected from marginal ears veins using an 18 gauge needle, and then stored at 2-8° C. overnight to allow for clot shrinkage. The samples were then centrifuged (400×g) and the sera were removed by pipette and frozen as individual samples at −10° C. to −25° C. until assayed. The suspensions were injected with 1.0 ml dose volumes.

### Homogenisation of the Carrier Systems

High pressure homogenisation using micro fluidiser was used for the preparation of the complex combinations, pressure range: 170-2700 bar.

### Lyophilisation/Dehydration of the Carrier Systems

The appropriate volume of suspension was added into each vial in 2 ml increments with vigorous vortexing between additions. Lyophilisation took place using freeze drying technology controlling all aspects of the lyophilisation cycle. The 1 shelf temperature ranges from −70° C. to 60° C., process condenser temperature as low as −85° C. and vacuum indication of 760 torr to 1 militorr. The dehydration of the carrier system without the use of a protective sugar depends on the organosilicon, sugar organosilicon and/or amino sugar organosilicon compound concentrations. Dehydrated carrier system complexes are prepared by drying the preparations under reduced pressure in the presence of one or more protective sugars, e.g. disaccharides such as trehalose and sucrose. Sugar organosilicon or amino sugar organosilicon compounds could be used as protective compounds for dehydration and/or lyophilisation of the carrier systems.

The dehydration was performed to an end point which results in sufficient water being left in the preparation (e.g. at least 4-10 moles water/silicon-lipid) so that the integrity of a substantial portion of the silicon-lipid combinations is retained upon rehydration.

### Hydration of the Lyophilised Carrier Systems

Hydration is the final step in the preparation of the formulation for injection and/or other pharmaceutical formulations (for oral, nasal and/or topical administration). Typically, hydration solution is added in a series of aliquots to lyophilised carrier systems with vortex mixing after each addition of hydration media. Generally, water for injection (WFI) is used. Here, we also tested WFI that was supplemented with 5% ethanol (EtOH), which has the added advantage of enhancing hydration of the carrier systems.

### Extrusion of the Carrier System Suspensions

Virtually all organosilicon, sugar organosilicon and/or amino sugar organosilicon compound complexes with proteins, allergens, and biological mixtures can be rapidly extruded resulting in a homogenous formulation of the preparations. Avestin LiposoFast and Northern Lipids Extruders (Thermobarrel Extruder) equipment attached to a nitrogen gas line was used.

### Preparation of Dispersions

A dispersion is a homogenous mixture of substances that are not soluble in each other. Dispersion can be achieved by sonication, extrusion, high pressure homogenisation, shaking, freezing and thawing.

### Preparation of Organosilicons

Examples 1-3 describe the synthesis of a number of the organosilicons. This procedure was employed to prepare the organosilicons of interest.

### Preparation of Amino Sugar Organosilicon Compound

Example 4 describes the procedures used for the preparation of an example of the sugar organosilicons. Modified and/or different procedures have also been used for the synthesis of the other sugar organosilicon compounds.

### Preparation of Siosomes and Sugar Siosomes

Example 5 describes the procedure used for the preparation of both blank (unloaded) siosomes and/or sugar-siosomes. The blank (unloaded) siosomes have water encapsulated inside the Siosomes and between the lipid layers.

All of the carrier systems and methods disclosed and claimed herein can be made and executed without undue experimentation in light of the present disclosure. While the carrier systems and methods of this invention have been described in terms of preferred embodiments, it will be apparent to those of skill in the art that variations may be applied to the carrier systems and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Preparation of Di(Decanoyloxy)Dimethylsilane

0.012 mol dimethyldichlorosilane is added to 50 ml anhydrous ether, to which is further added 0.02 mol sodium decanoate under agitation at 40° C. To increase the yield, an excess of dimethyl dichlorosilane is added. This is followed by approximately another 3 hours of agitation at 40° C. For hydrolysis of the excess dimethyldichlorosilane, water is added and approximately 10 ml ether is applied for extraction 3 to 5 times. The combined ether extracts are dried over anhydrous sodium sulphate and, after filtering off, evaporated in a vacuum. The remaining di(decanoyloxy)dimethylsilane is recrystallised from heptane.

Molecular formula: C₂₂H₄₄O₄Si
Molecular mass: 400.4 gmol⁻¹
Melting point: 32-33° C.

### Yield 92%

### Example 2

### Preparation of Di(Octadecanoloxy)Dimethylsilane

0.012 mol dimethyldichlorosilane is added to 50 ml anhydrous ether, to which 0.02 mol sodium octanoate is further added, under agitation at 40° C. This is followed by approximately 3 hours of further agitation at the same temperature. Disintegration of the excess dimethyldichlorosilane in water is carried out as per example 1. Recrystallisation from heptane is then performed.

Molecular formula: C₃₆H₇₆O₄Si
Molecular mass: 624.7 gmol⁻¹
Melting point: 62-64° C.

### Yield 68%

### Example 3

### Preparation of Di(Octanoloxy)Diphenylsilane

0.01 mol dichlorophenyl saline is added to 50 ml anhydrous ether, to which 0.02 mol sodium octanoate is further added under agitation at 40° C. This is followed by approximately another 5 hours of agitation, after which water is added and approximately 10 ml of ether is applied for extraction three times. The combined extracts are dried over sodium sulphate and evaporated in a vacuum.

Molecular formula: C₂₈H₄₀O₄Si
Molecular mass: 468.4 gmol⁻¹
Melting point 88-90° C.

### Yield 88%

### Example 4

### Preparation of Amino Sugar Organosilicon Compound

Synthesis of tetradecyl-(2,3,4,5-tetra-O-acetyl-β-D-gluco-pyranosyloxy)silanes. There exist various possibilities for the formation of glucosidic bonds to the anomeric C-atom of the sugar, as described in the literature. To the most important variants belong to the Konigs-Knorr reaction, the trichlorine-acetamidate-method and the TMSOTf method. All known methods are essentially S_{N}2-reactions, and through the right selection of the protective groups at the carbohydrate part and observance of suitable conditions of reaction, we obtained 1,2-trans-tied-glucosides. A further requirement is that the anomeric C atom must be activated through a leaving group which determines the centre of the reaction. The aglycon must have one free hydroxyl group if necessary selectively non-blocked.

### Example 5

### Preparation of Siosomes

10 μmol di(decanoyloxy)dimethylsilane is dissolved in 2 ml ethanol, and small doses age gradually added over a period of 4 hours to an aqueous phase (volume 2 ml) which can contain the compounds to be encapsulated and has been brought to a temperature of 80° C. Depending on the solubility of the compound to be encapsulated, it can also be a component of the organic phase. The material is the injected into pure water. In this case the agitation rate is 2000±200 rpm. The encapsulated compounds are separated from the non-encapsulated compounds by dialysis, gel filtration or centrifuging, for example and the contents of the siosomes determined. The siosomes prepared in this manner had a uniform size of 300 to 500 nm and are evidently all uni-lamellar. The siosomes stability was good.

The higher the number of side chain (from C=8 to C=18), the larger the siosomes become (200 nm to 2 μm). While in the case of di(octanoyloxy)dimethylsilane and di(decanoyloxy)dimethylsilane, nearly all the siosomes produced are unilamellar and of uniform size, other siosomes prepared according to the invention (with the number of carbon atoms in side chain C=12 to C=18) have predominantly multilamellar structures. These siosomes have a size of up to 2 μm.

### Example 6

### Preparation of a Carrier System Complex of Amino Sugar Organosilicon Compound with Insulin

A dispersion of 10 μmol of Didodecylsilyl-di(2,3,4,6-O-tetraacetyl-β-D-glucopyranosid) as the representative of amino sugar organosilicon compound, 0.01 M Tris/HCl, pH 7.4 and an aqueous solution of insulin (10 μmol, volume 3 ml) as antigen was prepared by mixing with a high pressure homogeniser. The homogenisation time may vary. The mixture was then incubated at 37° C. for 30 minutes and sterile filtrated and lyophilised at the temperature of −70° C. The lyophilised insulin-amino sugar organosilicon complex was reconstituted by hydration in sterile (deionized) water and the carrier system solution was used for in-vitro investigations.

After reconstitution by rehydration, the said carrier system surprisingly retains in solution more than 95% of the biological activity of insulin. The yield of the preparation process resulted from 3 independent experiments, and was 80-90% of the starting concentrations. Stability tests performed at 0, 4, 8, 12 and 48 hours at 4° C., 25° C. and 37° C. have shown that the carrier system complex of organosilicon-insulin is surprisingly stable and retains its biological activity.

### Example 7

### Preparation of a Carrier System Comprising Blank (Unloaded) Siosomes with Antigens

A dispersion of lyophilised blank sugar-siosomes prepared from 2-(dimethyldodecosilyl)-2,3,4,6-tetra-o-acetyl-β-D-glucopyranosid and 0.01 M Tris/HCl, pH7.4 and an aqueous solution of the antigen mixture comprising of Cathepsin B (EC 3.4.22.1), MW 30 kDa as Thiolproteinease and Cathepsin C (EC 3.4.14.1), MW 200 kDa as multimeric, dipeptidyl peptidase 1 enzyme was prepared at 37° C. by vortexing and high pressure homogenisation.

Directly afterward, the said dispersion was heated for 20 minutes at 25° C. (until complete fluidisation of the blank siosome). The mixture was then lyophilised and stored at 4-8° C. The biological activities of Cathepsin B and C in the carrier system complex were determined after reconstitution of the lyophilised samples by hydration with sterile (deionized) water using Cathepsin Activity Assay Kit.

Surprisingly, the carrier system retains more than 95% of the biologically active principles in solution. The carrier system showed long-termed stability as a lyophilisate at 4-8° C. The use of simple procedures in creating carrier systems, with the antigens Cathepsin B and C as examples for proteins, with lyophilised blank (unloaded) sugar-siosomes, demonstrates the potential for scale-up preparation of larger amounts.

### Example 8

### Preparation of a Carrier System Comprising Sugar Organosilicon Compounds and Blank (Unloaded) Sugar-Siosomes with an Allergen Mixture

A dispersion of blank (unloaded) sugar-siosomes as lyophilised powder with a particle size of 100-600 nm (following the rehydration with sterile water) was prepared from didodecylsilyl-bis(2,3,4,6-tetra-o-acetyl-β-D-glucopyranosid) and the lyophilised powder of a purified suspension of a mixture of the following antigens using Ultraturrax-type homogeniser at 4° C.:

Cathepsin B (EC 3.4.22.1), MW 30 kDa, as Thiolproteinase; Cathepsin D (EC 3.4.23.5), MW 42 kDa, as lysosomal protease; Cathepsin L (EC 3.4.22), MW 23-24 kDa, as lysosomal endo-proteinase; and Cathepsin C (EC 3.4.14.1), MW 200 kDa, as multimetric dipeptidyl peptidase 1.

Tris-buffer was added at pH 7.4 and the temperature was kept at 4° C. for 30 minutes. The whole suspension was then stirred for one hour at low temperature (4° C.). Subsequently the suspension was homogenised then sterile filtrated and lyophilised at −70° C.

Surprisingly, after reconstitution of the lyophilisate of the carrier system of the blank sugar-siosomes with the antigen mixture of Cathepsin B, D, L and C, the carrier system retained in solution more than 95% of the biological activity of each of the used Cathepsin. These determinations were performed using the Cathepsin Activity

Assay Kit. The preparation process of the carrier system with the antigen mixture showed very high reproducibility and yield. In comparison, using encapsulation technology of the prior art it was not possible to encapsulate with high efficiency more than one Cathepsin in the siosomes using the same sugar organosilicon. The lyophilised carrier system showed long-term stability at 4° C.

### Example 9

### Preparation of a Carrier System Comprising Sugar Organosilicon Compounds and Blank (Unloaded) Sugar-Siosomes with Antigens

A dispersion of the amino sugar organosilicon compound 1-0-dimethyl (dodecyl)silyl-2,3,4,6-tetra-o-acetyl-β-D-glucopyranosid, and blank siosomes prepared from didodecylsilyl-bis(2,3,4,6-tetra-o-acetyl-β-D-glucopyranosid), buffer solution (HEPES 20 mM) [4-(2-hydroxyethyl)-1-piperazineethanesulfonic acid] and antigens trypsin (EC 3.4.21.4), MW 23.300 kDa, and chemotrypsin (EC 3.4.21.1), MW 25000 kDa, was prepared using an Ultraturrax-type homogeniser at 4° C.-37° C. for 30-90 minutes. The suspension was sterile filtrated through a membrane filter and lyophilised with and without additives, for example phosphate buffer. Surprisingly, the carrier system retains in solution after reconstitution of the lyophilised preparation more than 90% of the sera-proteinase activity of the Trypsin and chemotrypsin. The lyophilised carrier system was stable after reconstitution with water. The preparation process using the combination of sugar organosilicons and sugar-siosomes showed surprisingly very high reproducibility concerning complex formation, good yield of production under very mild conditions and represents a simple procedure. Two hydration solutions, including water and buffer (0.01 M Tris and HEPES), have been used to determine their effect upon immunogenicity and injection site reactogenicity. The hydration solution at pH7.4 showed the better reactogenicity with similar immunogenicity.

### Example 10

### Preparation of a Carrier System Comprising Sugar Organosilicon Compounds and Antigens

The above procedures described in example 9 were employed to prepare the carrier system in this example. According to these methods of preparation, a dispersion of the amino sugar organosilicon compound didodecylsilyl-bis(2,3,4,6-tetra-o-acetyl-β-D-glucopyranosid) and the antigens trypsin and chemotrypsin was prepared.

The lyophilised powders of the same sugar organosilicon compounds as in example 9 and blank siosomes prepared using the same sugar organosilicon compounds have been compared to each other for differences in the release of the antigens trypsin and chemotrypsin using in-vitro release models.

Unexpectedly, the carrier system prepared according to example 9 has shown a slower release profile of trypsin and chemotrypsin in comparison to the carrier system prepared according to example 10. The two carrier system preparations showed the same content/concentrations of the trypsin and chemotrypsin. This means they were pharmaceutically equivalent.

### Example 11

### Preparation of a Carrier System of Sugar Organosilicon Compound, Sugar-Siosomes and EGFR Antibody and/or EGFR Antibody Conjugates

The above procedures described in examples 6-10 were employed to prepare carrier systems of sugar organosilicons and sugar-siosomes with EGFR antibodies and EGFR antibody conjugates. Didodecylsilyl-bis(2,3,4,6-tetra-o-acetyl-β-D-glucopyranosid) was used in the example. The same sugar organosilicon compound has been used for the preparation of the sugar-siosomes.

The preparation process has shown surprisingly very high efficiency of the protein carrier system with an EGFR-antibody (>90%). This was calculated after centrifugation by quantification of the amount of the protein complex fraction and the free non-complexed protein present in the aqueous supernatant phase. The protein was quantified using a modified Lowry method. In addition, further investigations have been performed using EGFR as antigen. The EGFR-antibody retained its specificity to bind the EGFR (>90% of the starting specificity). The lyophilised powder showed good long term stability at 4-8° C. Carrier systems with the different sugar organosilicons and/or sugar-siosomes have shown unexpected differences in the release profile of the EGFR-antibody from the carrier system. The pH 7.4 condition has an unexpected positive effect on the stability of the suspension after the reconstitution of the lyophilisate by rehydration using buffer solution.

### Example 12

### Preparation of a Carrier System of Sugar Organosilicon Compound and Sugar-Siosomes with House Dust Mite Allergens

The above procedures described in Examples 6-11 were employed to prepare carrier systems comprising organosilicon, sugar organosilicon, and amino sugar organosilicon compounds with a mixture allergen extract of house dust mite (comprising the species Dermatophagoides pteronyssinus, Dermatophagoides farina, Blomia kulagini, Blomia tropicalis, Pyroglyphus africanus and Euroglyptus maynei).

The allergen extract of house dust mite used in the different examples is a natural extract. The bulk solution of the carrier system was filled into vaccine vials and lyophilised under sterile conditions. A number of investigations have been performed using the suspension after the reconstitution of the lyophilisate by rehydration with sterile water or buffer at pH 7.4.

Efficiency tests showed the preparation process of the carrier system using the procedures described in examples 6-11 showed surprisingly very high efficiency of the allergen complex formation with the house dust mite allergens (>90%). This was calculated after centrifugation by quantification of the amount of the protein complex fraction and the free non-complexed proteins present in the aqueous supernatant phase.

Immunogenicity tests showed surprisingly that no protein-protein, or allergen-allergen interactions took place during the preparation process of the carrier systems. This has been determined using 50 μg of Cathepsin B and Cathepsin C as reference allergens/proteins mixed with the house dust mite extract. The concentration and specificity of the two Cathepsins have been determined in the suspension of the carrier systems after the reconstitution of the lyophilisate by rehydration using sterile water or buffer at pH7.4 using the Cathepsin Activity Assay Kit. Immunogenicity tests were carried out according to the “In-vivo protocol for the immunogenicity test” using the suspension of the carrier system versus the natural extract of the house mite allergen. The results showed that the carrier system induced higher IgE antibodies than the house mite allergen extract.

The release profile of the allergens from the carrier system was tested. When analyzing the induction of IgE antibodies, the results showed that the carrier systems comprising different organosilicon, sugar organosilicon, or amino sugar organosilicon compounds had different profiles.

The stability of carrier system formulations of organosilicon, sugar organosilicon, amino sugar organosilicon compounds with house mite extracts is more stable at a broad range of temperatures in comparison to the house mite allergen extracts alone. The carrier system is unexpectedly easy to handle (storage, transport, safety and the risk of contamination).

### Example 13

### Reconstitution of the Prepared Carrier Systems for Use in Injections

The procedures described in examples 8-12 were employed for the preparation of the lyophilised carrier systems used in this example.

Sterile water has been used for all carrier systems. The injection site reaction grades were assessed visually in all rabbits after multiple injections. The data showed that the injection site reactions were minimal for all groups. Thus, visual assessment indicated that the carrier system preparations were very well tolerated when administered i.m.

The results showed that sterile water is appropriate for the reconstitution of the lyophilisate for clinical use and the low level of injection site reactions indicate that increased injection frequencies would likely be acceptable as a means of increasing the response levels while maintaining minimal injection site reactions.

Adjuvants such as glycosaminoglycan, and particularly hyaluronic acid, as bio-adhesive molecules on the surface of the carriers could have an impact on the mediation of cellular interactions that involve binding and entry into a cell. Sugars such as glucose, lactose, dextran, and disaccharide sugars such as trehalose and saccharose have been investigated as protective agents during the course of lyophilisation of carrier systems without sugar organosilicons.

A detergent such as Triton X-100 has been used in the preparation of the carrier systems of proteins from animal tissues and plants. The results showed that the use of non-ionic detergent is very useful for the isolation of membrane proteins and enables a high retention of protein activity. In the examples protein solubilizer Triton X-100 was used as sterile 10% Trito Ampules.

Buffer solutions at physiological pH 7.4 have been used in the different examples. The results showed effects on the solubility of the allergens, allergen conjugates, proteins, peptides, antibodies and on the reactogenicity in the injection site. Oil, such as Montanide, has been used in this example for the reconstitution of the carrier system lyophilisate. The suspensions have been injected to animals according to the “In-vivo protocol for the immunogenicity tests”. The results showed that no interaction between the carrier system and Montanide took place. Other oils such as sesame, olive, and sun flower oils have been used for the reconstitution of the carrier system lyophilisate. The suspensions have been injected into the animals according to the “In-vivo protocol for the immunogenicity tests”. The results showed that the oil suspension has different immunogenicity profile than the suspension in water or buffer. Furthermore, oil suspensions gave indications for an unexpected controlled release “Depot Effect”.

### DETAILED DESCRIPTION OF THE FIGURES

FIG. 1: Hydrophobic interactions between non-polar regions involved in the hydrophobic fatty acid chains of the organosilicon, sugar organosilicon, amino sugar organosilicon compounds and siosomes. Straight lines represent carbon chains, whereas R represents the polar head group.

FIG. 2: Hydrophobic interactions between organosilicon, sugar organosilicon, and amino sugar organosilicon compounds. Straight lines represent carbon chains, whereas Si represents the SI head groups.

FIG. 3: Some of the intermolecular forces involved in the interaction between organosilicon, sugar organosilicon, amino sugar organosilicon compounds and proteins. Represented are hydrogen bonding, hydrophobic interactions and dipole-instantaneous dipole interactions. Double-headed arrows represent attractive forces.

1. Carrier system for biological agents obtainable by
a) mixing one or more organosilicon compounds, selected from the group comprising:
organosilicon, sugar organosilicon, amino sugar organosilicon compounds, their derivatives, salts and/or the vesicles formed from them, with
one or more biological agents, selected from the group comprising:
Antigens, pre-antigens, antigen conjugates, antibodies, pre-antibodies, antibody conjugates, allergens, allergen extracts, nucleic acids, plasmids, proteins, peptides, pharmaceutical agents, immunologically active substances and/or cosmetics,
in solution at a pH value between 7 and 8, preferably 7.4, followed by
b) homogenisation or sonication of the mixture, followed by
c) sterile filtration of the mixture, followed by
d) lyophilisation.

2. Carrier system for biological agents according to claim 1, comprising organosilicon, sugar organosilicon, or aminosugar organosilicon compounds of the general formula 1: 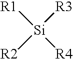
R1 and R2, which can be the same or different, each represent an acyloxy residue of the formula R—COO— or a peptide residue of the formula 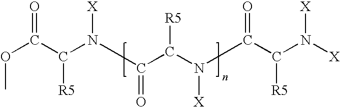
wherein R represents an unbranched or branched alkyl, alkenyl or alkinyl residue with 5 to 29 C atoms which can be substituted by one to three halogen atoms, alkoxy residues with 1 to 18 C atoms or amino groups, the residues R5, which can be the same or different, represent the residue remaining after the removal of the group 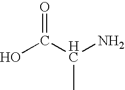
from an amino acid occurring in nature, the residues X, which may be the same or different, represent a hydrogen atom or an amino-protective group usually occurring in peptide chemistry and n represents an integer from 1 to 12 and R3 and R4 which can be the same or different, each represent an aryl group, such as a phenyl group, or the residue remaining after the removal of a hydrogen atom from a monosaccharide, disaccharide, amino sugar or a hydroxyl carbon acid, or alkoxy residues with 1-5 C atoms, or acyl residues of an amino acid occurring in nature with a free or protected amino group, or a dipeptide, tripeptide or tetrapeptide residue of an amino acid occurring in nature with free or protected amino groups, or they have the meaning R1 and R2, with R representing an unbranched or branched alkyl, akenyl or alkinyl residue.

3. Carrier system according to claim 1 or 2 comprising organosilicon, sugar organosilicon and/or amino sugar organosilicon compounds covalently attached to one or more of the antigens, antibodies and/or other biological agents.

4. Method for producing the carrier system according claim 1 or 2, comprising
a) mixing one or more organosilicon compounds, selected from the group comprising:
organosilicon, sugar organosilicon, amino sugar organosilicon compounds, their derivatives, salts and/or the vesicles formed from them, with
one or more biological agents, selected from the group comprising: antigens, pre-antigens, antigen conjugates, antibodies, pre-antibodies, antibody conjugates, allergens, allergen extracts, nucleic acids, plasmids, proteins, peptides, pharmaceutical agents, immunologically active substances and/or cosmetics,
in solution at a pH value between 7 and 8, preferably 7.4, followed by
b) homogenisation or sonication of the mixture, followed by
c) sterile filtration of the mixture, followed by
d) lyophilisation.

5. Method of claim 4, wherein the method is carried out between 4° C. and 37° C., particularly at 4° C.

6. The carrier system obtainable by the production method according to claim 4.

7. The carrier system according to claim 1 or 2, wherein said carrier system is a targeting system for biological agents.

8. The carrier system according to claim 1 or 2 wherein said carrier system is part of a cancer diagnostics.

9. Method for producing a pharmaceutical composition for the treatment of cancer comprising providing a carrier system according to claim 1 or 2 and producing said pharmaceutical composition.

10. The carrier system according to claim 1 or 2 wherein said carrier system is a constituent in a vaccine for therapeutic or prophylactic vaccination in humans and higher animals.

11. The carrier system according to claim 1 or 2 wherein said carrier system is a constituent in an immunotherapeutic for the desensitization of allergies in humans and higher animals.

12. The carrier system according to claim 1 or 2 wherein the carrier system contains one or more house dust mite allergens as immunotherapeutic and/or vaccine for the prevention and/or treatment of allergic disorders caused by house dust mite allergens, including anaphylaxis, seasonal hay fever, atopic dermatitis and allergic asthma.

13. Method for producing a pharmaceutical composition for the treatment of infectious, acute, chronic and life threatening diseases comprising providing a carrier system according to claim 1 or 2 and producing said pharmaceutical composition for the treatment of infectious, acute, chronic and life threatening diseases.

14. Method for preparing cosmetics comprising providing a carrier system according to claim 1 or 2 and preparing said cosmetics.

15. A kit for preparing the carrier system according to claim 1 or 2, wherein the silicon organic compound, sugar organosilicon and/or amino sugar organosilicon compounds, its derivatives, vesicles prepared from them are stored in a separate container from the biological agents, antigens, pre-antigens, antigen conjugates, antibodies, pre-antibodies, antibody conjugates, allergens, allergen extracts, nucleic acids, plasmids, proteins, peptides, pharmaceutical agents, immunologically active substances, cosmetics, additives, adjuvants, and solvents for reconstitution such as sterile water, detergent buffer or oil, allowing the administration of different concentrations and combinations of various agents, and wherein information about using parts of the kit is provided.
